SEQUENCE LISTING (313 sequences)

SEQ ID NO: 1 - DNA, 10 bp, Synthetic construct
  source 1..10: mol_type = other DNA
aattaagcgg

SEQ ID NO: 2 - DNA, 10 bp, Synthetic construct
  source 1..10: mol_type = other DNA
agacgaggtc

SEQ ID NO: 3 - DNA, 10 bp, Synthetic construct
  source 1..10: mol_type = other DNA
ctaataggct

SEQ ID NO: 4 - DNA, 10 bp, Synthetic construct
  source 1..10: mol_type = other DNA
gcctaccgat

SEQ ID NO: 5 - DNA, 10 bp, Synthetic construct
  source 1..10: mol_type = other DNA
cttatcaggt

SEQ ID NO: 6 - DNA, 10 bp, Synthetic construct
  source 1..10: mol_type = other DNA
tgcatatagg

SEQ ID NO: 7 - DNA, 10 bp, Synthetic construct
  source 1..10: mol_type = other DNA
gcacctgatc

SEQ ID NO: 8 - DNA, 10 bp, Synthetic construct
  source 1..10: mol_type = other DNA
gtactcgtga

SEQ ID NO: 9 - DNA, 10 bp, Synthetic construct
  source 1..10: mol_type = other DNA
cacatatgca

SEQ ID NO: 10 - DNA, 10 bp, Synthetic construct
  source 1..10: mol_type = other DNA
cgtactatac

SEQ ID NO: 11 - DNA, 10 bp, Synthetic construct
  source 1..10: mol_type = other DNA
agtgttgtct

SEQ ID NO: 12 - DNA, 10 bp, Synthetic construct
  source 1..10: mol_type = other DNA
cggttgccta

SEQ ID NO: 13 - DNA, 10 bp, Synthetic construct
  source 1..10: mol_type = other DNA
tatcagccaa

SEQ ID NO: 14 - DNA, 10 bp, Synthetic construct
  source 1..10: mol_type = other DNA
ccaagccatc

SEQ ID NO: 15 - DNA, 10 bp, Synthetic construct
  source 1..10: mol_type = other DNA
gatcgcctca

SEQ ID NO: 16 - DNA, 10 bp, Synthetic construct
  source 1..10: mol_type = other DNA
gctctgaagg

SEQ ID NO: 17 - DNA, 10 bp, Synthetic construct
  source 1..10: mol_type = other DNA
cttggcctct

SEQ ID NO: 18 - DNA, 10 bp, Synthetic construct
  source 1..10: mol_type = other DNA
tcatcggaat

SEQ ID NO: 19 - DNA, 10 bp, Synthetic construct
  source 1..10: mol_type = other DNA
tcaccgtata

SEQ ID NO: 20 - DNA, 10 bp, Synthetic construct
  source 1..10: mol_type = other DNA
tgttacctca

SEQ ID NO: 21 - DNA, 10 bp, Synthetic construct
  source 1..10: mol_type = other DNA
gaaggcctaa

SEQ ID NO: 22 - DNA, 10 bp, Synthetic construct
  source 1..10: mol_type = other DNA
tacgaatcga

SEQ ID NO: 23 - DNA, 10 bp, Synthetic construct
  source 1..10: mol_type = other DNA
cgaatggtaa

SEQ ID NO: 24 - DNA, 10 bp, Synthetic construct
  source 1..10: mol_type = other DNA
ccgcaatagg

SEQ ID NO: 25 - DNA, 10 bp, Synthetic construct
  source 1..10: mol_type = other DNA
tgttagaggt

SEQ ID NO: 26 - DNA, 10 bp, Synthetic construct
  source 1..10: mol_type = other DNA
gttcctcatt

SEQ ID NO: 27 - DNA, 10 bp, Synthetic construct
  source 1..10: mol_type = other DNA
ctggtattag

SEQ ID NO: 28 - DNA, 10 bp, Synthetic construct
  source 1..10: mol_type = other DNA
aggcctccaa

SEQ ID NO: 29 - DNA, 10 bp, Synthetic construct
  source 1..10: mol_type = other DNA
tcggctatga

SEQ ID NO: 30 - DNA, 10 bp, Synthetic construct
  source 1..10: mol_type = other DNA
gcgagccata

SEQ ID NO: 31 - DNA, 10 bp, Synthetic construct
  source 1..10: mol_type = other DNA
ccttcatagg

SEQ ID NO: 32 - DNA, 10 bp, Synthetic construct
  source 1..10: mol_type = other DNA
gcttgagacg

SEQ ID NO: 33 - DNA, 10 bp, Synthetic construct
  source 1..10: mol_type = other DNA
gaggagtatg

SEQ ID NO: 34 - DNA, 10 bp, Synthetic construct
  source 1..10: mol_type = other DNA
gcttacactt

SEQ ID NO: 35 - DNA, 10 bp, Synthetic construct
  source 1..10: mol_type = other DNA
ctgtgcgtag

SEQ ID NO: 36 - DNA, 10 bp, Synthetic construct
  source 1..10: mol_type = other DNA
ttgtggcgca

SEQ ID NO: 37 - DNA, 10 bp, Synthetic construct
  source 1..10: mol_type = other DNA
gtcgatgatt

SEQ ID NO: 38 - DNA, 10 bp, Synthetic construct
  source 1..10: mol_type = other DNA
acgcgagtca

SEQ ID NO: 39 - DNA, 10 bp, Synthetic construct
  source 1..10: mol_type = other DNA
ctgtgaagaa

SEQ ID NO: 40 - DNA, 10 bp, Synthetic construct
  source 1..10: mol_type = other DNA
tacgcaacgg

SEQ ID NO: 41 - DNA, 10 bp, Synthetic construct
  source 1..10: mol_type = other DNA
tccactgcgt

SEQ ID NO: 42 - DNA, 10 bp, Synthetic construct
  source 1..10: mol_type = other DNA
ttgagcctgg

SEQ ID NO: 43 - DNA, 10 bp, Synthetic construct
  source 1..10: mol_type = other DNA
tagtgaatcg

SEQ ID NO: 44 - DNA, 10 bp, Synthetic construct
  source 1..10: mol_type = other DNA
gcctgtactg

SEQ ID NO: 45 - DNA, 10 bp, Synthetic construct
  source 1..10: mol_type = other DNA
gtgataagct

SEQ ID NO: 46 - DNA, 10 bp, Synthetic construct
  source 1..10: mol_type = other DNA
ctaccacgaa

SEQ ID NO: 47 - DNA, 10 bp, Synthetic construct
  source 1..10: mol_type = other DNA
aaggcaagtt

SEQ ID NO: 48 - DNA, 10 bp, Synthetic construct
  source 1..10: mol_type = other DNA
ttctagatgg

SEQ ID NO: 49 - DNA, 10 bp, Synthetic construct
  source 1..10: mol_type = other DNA
accactgtta

SEQ ID NO: 50 - DNA, 10 bp, Synthetic construct
  source 1..10: mol_type = other DNA
gcaccgctat

SEQ ID NO: 51 - DNA, 10 bp, Synthetic construct
  source 1..10: mol_type = other DNA
tagtaaccga

SEQ ID NO: 52 - DNA, 10 bp, Synthetic construct
  source 1..10: mol_type = other DNA
ttgctcacca

SEQ ID NO: 53 - DNA, 19 bp, Synthetic construct
  source 1..19: mol_type = other DNA
  modified_base 4: mod_base = OTHER; note = "t" is deoxyuracil
  modified_base 9: mod_base = OTHER; note = "t" is deoxyuracil
  modified_base 12: mod_base = OTHER; note = "t" is deoxyuracil
  modified_base 17: mod_base = OTHER; note = "t" is deoxyuracil
ttctagccttctcgcatca

SEQ ID NO: 54 - DNA, 26 bp, Synthetic construct
  source 1..26: mol_type = other DNA
ttccctacacgacgctcttccgatct

SEQ ID NO: 55 - DNA, 10 bp, Synthetic construct
  source 1..10: mol_type = other DNA
atacacatct

SEQ ID NO: 56 - DNA, 19 bp, Synthetic construct
  source 1..19: mol_type = other DNA
agatgtgtataagagacag

SEQ ID NO: 57 - DNA, 25 bp, Synthetic construct
  source 1..25: mol_type = other DNA
cagtgcagatgtgtataagagacag

SEQ ID NO: 58 - DNA, 19 bp, Synthetic construct
  source 1..19: mol_type = other DNA
ctgtctcttatacacatct

SEQ ID NO: 59 - DNA, 52 bp, Synthetic construct
  source 1..52: mol_type = other DNA
gcactgtcggttactattctagccttctcgcatcatacatcgtagtaaccga

SEQ ID NO: 60 - DNA, 35 bp, Synthetic construct
  source 1..35: mol_type = other DNA
  modified_base 5: mod_base = OTHER; note = "t" is deoxyuracil
  modified_base 11: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgccgcttaattttctagccttctcgcatca

SEQ ID NO: 61 - DNA, 35 bp, Synthetic construct
  source 1..35: mol_type = other DNA
  modified_base 5: mod_base = OTHER; note = "t" is deoxyuracil
  modified_base 11: mod_base = OTHER; note = "t" is deoxyuracil
  modified_base 14: mod_base = OTHER; note = "t" is deoxyuracil
  modified_base 16: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactggacctcgtctttctagccttctcgcatca

SEQ ID NO: 62 - DNA, 35 bp, Synthetic construct
  source 1..35: mol_type = other DNA
  modified_base 5: mod_base = OTHER; note = "t" is deoxyuracil
  modified_base 11: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgagcctattagttctagccttctcgcatca

SEQ ID NO: 63 - DNA, 35 bp, Synthetic construct
  source 1..35: mol_type = other DNA
  modified_base 5: mod_base = OTHER; note = "t" is deoxyuracil
  modified_base 8: mod_base = OTHER; note = "t" is deoxyuracil
  modified_base 12: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgatcggtaggcttctagccttctcgcatca

SEQ ID NO: 64 - DNA, 35 bp, Synthetic construct
  source 1..35: mol_type = other DNA
  modified_base 5: mod_base = OTHER; note = "t" is deoxyuracil
  modified_base 10: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgacctgataagttctagccttctcgcatca

SEQ ID NO: 65 - DNA, 35 bp, Synthetic construct
  source 1..35: mol_type = other DNA
  modified_base 5: mod_base = OTHER; note = "t" is deoxyuracil
  modified_base 9: mod_base = OTHER; note = "t" is deoxyuracil
  modified_base 11: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgcctatatgcattctagccttctcgcatca

SEQ ID NO: 66 - DNA, 35 bp, Synthetic construct
  source 1..35: mol_type = other DNA
  modified_base 5: mod_base = OTHER; note = "t" is deoxyuracil
  modified_base 9: mod_base = OTHER; note = "t" is deoxyuracil
  modified_base 14: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactggatcaggtgcttctagccttctcgcatca

SEQ ID NO: 67 - DNA, 35 bp, Synthetic construct
  source 1..35: mol_type = other DNA
  modified_base 5: mod_base = OTHER; note = "t" is deoxyuracil
  modified_base 7: mod_base = OTHER; note = "t" is deoxyuracil
  modified_base 14: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgtcacgagtacttctagccttctcgcatca

SEQ ID NO: 68 - DNA, 35 bp, Synthetic construct
  source 1..35: mol_type = other DNA
  modified_base 5: mod_base = OTHER; note = "t" is deoxyuracil
  modified_base 7: mod_base = OTHER; note = "t" is deoxyuracil
  modified_base 11: mod_base = OTHER; note = "t" is deoxyuracil
  modified_base 13: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgtgcatatgtgttctagccttctcgcatca

SEQ ID NO: 69 - DNA, 35 bp, Synthetic construct
  source 1..35: mol_type = other DNA
  modified_base 5: mod_base = OTHER; note = "t" is deoxyuracil
  modified_base 8: mod_base = OTHER; note = "t" is deoxyuracil
  modified_base 10: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactggtatagtacgttctagccttctcgcatca

SEQ ID NO: 70 - DNA, 35 bp, Synthetic construct
  source 1..35: mol_type = other DNA
  modified_base 5: mod_base = OTHER; note = "t" is deoxyuracil
  modified_base 16: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgagacaacactttctagccttctcgcatca

SEQ ID NO: 71 - DNA, 35 bp, Synthetic construct
  source 1..35: mol_type = other DNA
  modified_base 5: mod_base = OTHER; note = "t" is deoxyuracil
  modified_base 7: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
gcactgtaggcaaccgttctagccttctcgcatca

SEQ ID NO: 72 - DNA, 16 bp, Synthetic construct
  source 1..16: mol_type = other DNA
acatcgaattaagcgg

SEQ ID NO: 73 - DNA, 16 bp, Synthetic construct
  source 1..16: mol_type = other DNA
acatcgagacgaggtc

SEQ ID NO: 74 - DNA, 16 bp, Synthetic construct
  source 1..16: mol_type = other DNA
acatcgctaataggct

SEQ ID NO: 75 - DNA, 16 bp, Synthetic construct
  source 1..16: mol_type = other DNA
acatcggcctaccgat

SEQ ID NO: 76 - DNA, 16 bp, Synthetic construct
  source 1..16: mol_type = other DNA
acatcgcttatcaggt

SEQ ID NO: 77 - DNA, 16 bp, Synthetic construct
  source 1..16: mol_type = other DNA
acatcgtgcatatagg

SEQ ID NO: 78 - DNA, 16 bp, Synthetic construct
  source 1..16: mol_type = other DNA
acatcggcacctgatc

SEQ ID NO: 79 - DNA, 16 bp, Synthetic construct
  source 1..16: mol_type = other DNA
acatcggtactcgtga

SEQ ID NO: 80 - DNA, 16 bp, Synthetic construct
  source 1..16: mol_type = other DNA
acatcgcacatatgca

SEQ ID NO: 81 - DNA, 16 bp, Synthetic construct
  source 1..16: mol_type = other DNA
acatcgcgtactatac

SEQ ID NO: 82 - DNA, 16 bp, Synthetic construct
  source 1..16: mol_type = other DNA
acatcgagtgttgtct

SEQ ID NO: 83 - DNA, 16 bp, Synthetic construct
  source 1..16: mol_type = other DNA
acatcgcggttgccta

SEQ ID NO: 84 - DNA, 10 bp, Synthetic construct
  source 1..10: mol_type = other DNA
  modified_base 5: mod_base = OTHER; note = "t" is deoxyuracil
  modified_base 9: mod_base = OTHER; note = "t" is deoxyuracil
ccgcttaatt

SEQ ID NO: 85 - DNA, 10 bp, Synthetic construct
  source 1..10: mol_type = other DNA
  modified_base 5: mod_base = OTHER; note = "t" is deoxyuracil
  modified_base 8: mod_base = OTHER; note = "t" is deoxyuracil
  modified_base 10: mod_base = OTHER; note = "t" is deoxyuracil
gacctcgtct

SEQ ID NO: 86 - DNA, 10 bp, Synthetic construct
  source 1..10: mol_type = other DNA
  modified_base 5: mod_base = OTHER; note = "t" is deoxyuracil
  modified_base 7: mod_base = OTHER; note = "t" is deoxyuracil
agcctattag

SEQ ID NO: 87 - DNA, 10 bp, Synthetic construct
  source 1..10: mol_type = other DNA
  modified_base 2: mod_base = OTHER; note = "t" is deoxyuracil
  modified_base 6: mod_base = OTHER; note = "t" is deoxyuracil
atcggtaggc

SEQ ID NO: 88 - DNA, 10 bp, Synthetic construct
  source 1..10: mol_type = other DNA
  modified_base 4: mod_base = OTHER; note = "t" is deoxyuracil
  modified_base 7: mod_base = OTHER; note = "t" is deoxyuracil
acctgataag

SEQ ID NO: 89 - DNA, 10 bp, Synthetic construct
  source 1..10: mol_type = other DNA
  modified_base 3: mod_base = OTHER; note = "t" is deoxyuracil
  modified_base 5: mod_base = OTHER; note = "t" is deoxyuracil
  modified_base 7: mod_base = OTHER; note = "t" is deoxyuracil
cctatatgca

SEQ ID NO: 90 - DNA, 10 bp, Synthetic construct
  source 1..10: mol_type = other DNA
  modified_base 3: mod_base = OTHER; note = "t" is deoxyuracil
  modified_base 8: mod_base = OTHER; note = "t" is deoxyuracil
gatcaggtgc

SEQ ID NO: 91 - DNA, 10 bp, Synthetic construct
  source 1..10: mol_type = other DNA
  modified_base 1: mod_base = OTHER; note = "t" is deoxyuracil
  modified_base 8: mod_base = OTHER; note = "t" is deoxyuracil
tcacgagtac

SEQ ID NO: 92 - DNA, 10 bp, Synthetic construct
  source 1..10: mol_type = other DNA
  modified_base 1: mod_base = OTHER; note = "t" is deoxyuracil
  modified_base 5: mod_base = OTHER; note = "t" is deoxyuracil
  modified_base 7: mod_base = OTHER; note = "t" is deoxyuracil
  modified_base 9: mod_base = OTHER; note = "t" is deoxyuracil
tgcatatgtg

SEQ ID NO: 93 - DNA, 10 bp, Synthetic construct
  source 1..10: mol_type = other DNA
  modified_base 2: mod_base = OTHER; note = "t" is deoxyuracil
  modified_base 4: mod_base = OTHER; note = "t" is deoxyuracil
  modified_base 7: mod_base = OTHER; note = "t" is deoxyuracil
gtatagtacg

SEQ ID NO: 94 - DNA, 10 bp, Synthetic construct
  source 1..10: mol_type = other DNA
  modified_base 10: mod_base = OTHER; note = "t" is deoxyuracil
agacaacact

SEQ ID NO: 95 - DNA, 10 bp, Synthetic construct
  source 1..10: mol_type = other DNA
  modified_base 1: mod_base = OTHER; note = "t" is deoxyuracil
taggcaaccg

SEQ ID NO: 96 - DNA, 51 bp, Synthetic construct
  source 1..51: mol_type = other DNA
cgatgttaacagtggtttctagccttctcgcatcatcgtctaccactgtta

SEQ ID NO: 97 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 8: mod_base = OTHER; note = "t" is deoxyuracil
  modified_base 12: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtttggctgatattctagccttctcgcatca

SEQ ID NO: 98 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 9: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtgatggcttggttctagccttctcgcatca

SEQ ID NO: 99 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgttgaggcgatcttctagccttctcgcatca

SEQ ID NO: 100 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 9: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtccttcagagcttctagccttctcgcatca

SEQ ID NO: 101 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtagaggccaagttctagccttctcgcatca

SEQ ID NO: 102 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 8: mod_base = OTHER; note = "t" is deoxyuracil
  modified_base 14: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtattccgatgattctagccttctcgcatca

SEQ ID NO: 103 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 9: mod_base = OTHER; note = "t" is deoxyuracil
  modified_base 14: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgttatacggtgattctagccttctcgcatca

SEQ ID NO: 104 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 12: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgttgaggtaacattctagccttctcgcatca

SEQ ID NO: 105 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 8: mod_base = OTHER; note = "t" is deoxyuracil
  modified_base 14: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtttaggccttcttctagccttctcgcatca

SEQ ID NO: 106 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 11: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgttcgattcgtattctagccttctcgcatca

SEQ ID NO: 107 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 8: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtttaccattcgttctagccttctcgcatca

SEQ ID NO: 108 - DNA, 35 bp, Synthetic construct
  source 1..35: mol_type = other DNA
  modified_base 4: mod_base = OTHER; note = "t" is deoxyuracil
  modified_base 6: mod_base = OTHER; note = "t" is deoxyuracil
  modified_base 9: mod_base = OTHER; note = "t" is deoxyuracil
  modified_base 11: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
cgatgtcctattgcggttctagccttctcgcatca

SEQ ID NO: 109 - DNA, 16 bp, Synthetic construct
  source 1..16: mol_type = other DNA
tcgtcttatcagccaa

SEQ ID NO: 110 - DNA, 16 bp, Synthetic construct
  source 1..16: mol_type = other DNA
tcgtctccaagccatc

SEQ ID NO: 111 - DNA, 16 bp, Synthetic construct
  source 1..16: mol_type = other DNA
tcgtctgatcgcctca

SEQ ID NO: 112 - DNA, 16 bp, Synthetic construct
  source 1..16: mol_type = other DNA
tcgtctgctctgaagg

SEQ ID NO: 113 - DNA, 16 bp, Synthetic construct
  source 1..16: mol_type = other DNA
tcgtctcttggcctct

SEQ ID NO: 114 - DNA, 16 bp, Synthetic construct
  source 1..16: mol_type = other DNA
tcgtcttcatcggaat

SEQ ID NO: 115 - DNA, 16 bp, Synthetic construct
  source 1..16: mol_type = other DNA
tcgtcttcaccgtata

SEQ ID NO: 116 - DNA, 16 bp, Synthetic construct
  source 1..16: mol_type = other DNA
tcgtcttgttacctca

SEQ ID NO: 117 - DNA, 16 bp, Synthetic construct
  source 1..16: mol_type = other DNA
tcgtctgaaggcctaa

SEQ ID NO: 118 - DNA, 16 bp, Synthetic construct
  source 1..16: mol_type = other DNA
tcgtcttacgaatcga

SEQ ID NO: 119 - DNA, 16 bp, Synthetic construct
  source 1..16: mol_type = other DNA
tcgtctcgaatggtaa

SEQ ID NO: 120 - DNA, 16 bp, Synthetic construct
  source 1..16: mol_type = other DNA
tcgtctccgcaatagg

SEQ ID NO: 121 - DNA, 10 bp, Synthetic construct
  source 1..10: mol_type = other DNA
  modified_base 2: mod_base = OTHER; note = "t" is deoxyuracil
  modified_base 6: mod_base = OTHER; note = "t" is deoxyuracil
  modified_base 9: mod_base = OTHER; note = "t" is deoxyuracil
ttggctgata

SEQ ID NO: 122 - DNA, 10 bp, Synthetic construct
  source 1..10: mol_type = other DNA
  modified_base 3: mod_base = OTHER; note = "t" is deoxyuracil
  modified_base 7: mod_base = OTHER; note = "t" is deoxyuracil
gatggcttgg

SEQ ID NO: 123 - DNA, 10 bp, Synthetic construct
  source 1..10: mol_type = other DNA
  modified_base 9: mod_base = OTHER; note = "t" is deoxyuracil
tgaggcgatc

SEQ ID NO: 124 - DNA, 10 bp, Synthetic construct
  source 1..10: mol_type = other DNA
  modified_base 3: mod_base = OTHER; note = "t" is deoxyuracil
ccttcagagc

SEQ ID NO: 125 - DNA, 10 bp, Synthetic construct
  source 1..10: mol_type = other DNA
agaggccaag

SEQ ID NO: 126 - DNA, 10 bp, Synthetic construct
  source 1..10: mol_type = other DNA
  modified_base 2: mod_base = OTHER; note = "t" is deoxyuracil
  modified_base 8: mod_base = OTHER; note = "t" is deoxyuracil
attccgatga

SEQ ID NO: 127 - DNA, 10 bp, Synthetic construct
  source 1..10: mol_type = other DNA
  modified_base 3: mod_base = OTHER; note = "t" is deoxyuracil
  modified_base 8: mod_base = OTHER; note = "t" is deoxyuracil
tatacggtga

SEQ ID NO: 128 - DNA, 10 bp, Synthetic construct
  source 1..10: mol_type = other DNA
  modified_base 6: mod_base = OTHER; note = "t" is deoxyuracil
tgaggtaaca

SEQ ID NO: 129 - DNA, 10 bp, Synthetic construct
  source 1..10: mol_type = other DNA
  modified_base 2: mod_base = OTHER; note = "t" is deoxyuracil
  modified_base 8: mod_base = OTHER; note = "t" is deoxyuracil
ttaggccttc

SEQ ID NO: 130 - DNA, 10 bp, Synthetic construct
  source 1..10: mol_type = other DNA
  modified_base 5: mod_base = OTHER; note = "t" is deoxyuracil
  modified_base 9: mod_base = OTHER; note = "t" is deoxyuracil
tcgattcgta

SEQ ID NO: 131 - DNA, 10 bp, Synthetic construct
  source 1..10: mol_type = other DNA
  modified_base 2: mod_base = OTHER; note = "t" is deoxyuracil
  modified_base 7: mod_base = OTHER; note = "t" is deoxyuracil
ttaccattcg

SEQ ID NO: 132 - DNA, 10 bp, Synthetic construct
  source 1..10: mol_type = other DNA
  modified_base 3: mod_base = OTHER; note = "t" is deoxyuracil
  modified_base 5: mod_base = OTHER; note = "t" is deoxyuracil
cctattgcgg

SEQ ID NO: 133 - DNA, 51 bp, Synthetic construct
  source 1..51: mol_type = other DNA
agacgaatagcggtgcttctagccttctcgcatcactctgtgcaccgctat

SEQ ID NO: 134 - DNA, 35 bp, Synthetic construct
  source 1..35: mol_type = other DNA
  modified_base 10: mod_base = OTHER; note = "t" is deoxyuracil
  modified_base 12: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgaacctctaacattctagccttctcgcatca

SEQ ID NO: 135 - DNA, 35 bp, Synthetic construct
  source 1..35: mol_type = other DNA
  modified_base 9: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgaaatgaggaacttctagccttctcgcatca

SEQ ID NO: 136 - DNA, 35 bp, Synthetic construct
  source 1..35: mol_type = other DNA
  modified_base 8: mod_base = OTHER; note = "t" is deoxyuracil
  modified_base 11: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgactaataccagttctagccttctcgcatca

SEQ ID NO: 137 - DNA, 35 bp, Synthetic construct
  source 1..35: mol_type = other DNA
  modified_base 7: mod_base = OTHER; note = "t" is deoxyuracil
  modified_base 16: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgattggaggcctttctagccttctcgcatca

SEQ ID NO: 138 - DNA, 35 bp, Synthetic construct
  source 1..35: mol_type = other DNA
  modified_base 7: mod_base = OTHER; note = "t" is deoxyuracil
  modified_base 10: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgatcatagccgattctagccttctcgcatca

SEQ ID NO: 139 - DNA, 35 bp, Synthetic construct
  source 1..35: mol_type = other DNA
  modified_base 7: mod_base = OTHER; note = "t" is deoxyuracil
  modified_base 9: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgatatggctcgcttctagccttctcgcatca

SEQ ID NO: 140 - DNA, 35 bp, Synthetic construct
  source 1..35: mol_type = other DNA
  modified_base 9: mod_base = OTHER; note = "t" is deoxyuracil
  modified_base 11: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgacctatgaaggttctagccttctcgcatca

SEQ ID NO: 141 - DNA, 35 bp, Synthetic construct
  source 1..35: mol_type = other DNA
  modified_base 9: mod_base = OTHER; note = "t" is deoxyuracil
  modified_base 11: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgacgtctcaagcttctagccttctcgcatca

SEQ ID NO: 142 - DNA, 35 bp, Synthetic construct
  source 1..35: mol_type = other DNA
  modified_base 9: mod_base = OTHER; note = "t" is deoxyuracil
  modified_base 12: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgacatactcctcttctagccttctcgcatca

SEQ ID NO: 143 - DNA, 35 bp, Synthetic construct
  source 1..35: mol_type = other DNA
  modified_base 10: mod_base = OTHER; note = "t" is deoxyuracil
  modified_base 12: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgaaagtgtaagcttctagccttctcgcatca

SEQ ID NO: 144 - DNA, 35 bp, Synthetic construct
  source 1..35: mol_type = other DNA
  modified_base 8: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgactacgcacagttctagccttctcgcatca

SEQ ID NO: 145 - DNA, 35 bp, Synthetic construct
  source 1..35: mol_type = other DNA
  modified_base 7: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
agacgatgcgccacaattctagccttctcgcatca

SEQ ID NO: 146 - DNA, 16 bp, Synthetic construct
  source 1..16: mol_type = other DNA
ctctgttgttagaggt

SEQ ID NO: 147 - DNA, 16 bp, Synthetic construct
  source 1..16: mol_type = other DNA
ctctgtgttcctcatt

SEQ ID NO: 148 - DNA, 16 bp, Synthetic construct
  source 1..16: mol_type = other DNA
ctctgtctggtattag

SEQ ID NO: 149 - DNA, 16 bp, Synthetic construct
  source 1..16: mol_type = other DNA
ctctgtaggcctccaa

SEQ ID NO: 150 - DNA, 16 bp, Synthetic construct
  source 1..16: mol_type = other DNA
ctctgttcggctatga

SEQ ID NO: 151 - DNA, 16 bp, Synthetic construct
  source 1..16: mol_type = other DNA
ctctgtgcgagccata

SEQ ID NO: 152 - DNA, 16 bp, Synthetic construct
  source 1..16: mol_type = other DNA
ctctgtccttcatagg

SEQ ID NO: 153 - DNA, 16 bp, Synthetic construct
  source 1..16: mol_type = other DNA
ctctgtgcttgagacg

SEQ ID NO: 154 - DNA, 16 bp, Synthetic construct
  source 1..16: mol_type = other DNA
ctctgtgaggagtatg

SEQ ID NO: 155 - DNA, 16 bp, Synthetic construct
  source 1..16: mol_type = other DNA
ctctgtgcttacactt

SEQ ID NO: 156 - DNA, 16 bp, Synthetic construct
  source 1..16: mol_type = other DNA
ctctgtctgtgcgtag

SEQ ID NO: 157 - DNA, 16 bp, Synthetic construct
  source 1..16: mol_type = other DNA
ctctgtttgtggcgca

SEQ ID NO: 158 - DNA, 10 bp, Synthetic construct
  source 1..10: mol_type = other DNA
  modified_base 4: mod_base = OTHER; note = "t" is deoxyuracil
  modified_base 6: mod_base = OTHER; note = "t" is deoxyuracil
acctctaaca

SEQ ID NO: 159 - DNA, 10 bp, Synthetic construct
  source 1..10: mol_type = other DNA
  modified_base 3: mod_base = OTHER; note = "t" is deoxyuracil
aatgaggaac

SEQ ID NO: 160 - DNA, 10 bp, Synthetic construct
  source 1..10: mol_type = other DNA
  modified_base 2: mod_base = OTHER; note = "t" is deoxyuracil
  modified_base 5: mod_base = OTHER; note = "t" is deoxyuracil
ctaataccag

SEQ ID NO: 161 - DNA, 10 bp, Synthetic construct
  source 1..10: mol_type = other DNA
  modified_base 1: mod_base = OTHER; note = "t" is deoxyuracil
  modified_base 10: mod_base = OTHER; note = "t" is deoxyuracil
ttggaggcct

SEQ ID NO: 162 - DNA, 10 bp, Synthetic construct
  source 1..10: mol_type = other DNA
  modified_base 1: mod_base = OTHER; note = "t" is deoxyuracil
  modified_base 4: mod_base = OTHER; note = "t" is deoxyuracil
tcatagccga

SEQ ID NO: 163 - DNA, 10 bp, Synthetic construct
  source 1..10: mol_type = other DNA
  modified_base 1: mod_base = OTHER; note = "t" is deoxyuracil
  modified_base 3: mod_base = OTHER; note = "t" is deoxyuracil
  modified_base 7: mod_base = OTHER; note = "t" is deoxyuracil
tatggctcgc

SEQ ID NO: 164 - DNA, 10 bp, Synthetic construct
  source 1..10: mol_type = other DNA
  modified_base 3: mod_base = OTHER; note = "t" is deoxyuracil
  modified_base 5: mod_base = OTHER; note = "t" is deoxyuracil
cctatgaagg

SEQ ID NO: 165 - DNA, 10 bp, Synthetic construct
  source 1..10: mol_type = other DNA
  modified_base 3: mod_base = OTHER; note = "t" is deoxyuracil
  modified_base 5: mod_base = OTHER; note = "t" is deoxyuracil
cgtctcaagc

SEQ ID NO: 166 - DNA, 10 bp, Synthetic construct
  source 1..10: mol_type = other DNA
  modified_base 3: mod_base = OTHER; note = "t" is deoxyuracil
  modified_base 6: mod_base = OTHER; note = "t" is deoxyuracil
  modified_base 9: mod_base = OTHER; note = "t" is deoxyuracil
catactcctc

SEQ ID NO: 167 - DNA, 10 bp, Synthetic construct
  source 1..10: mol_type = other DNA
  modified_base 4: mod_base = OTHER; note = "t" is deoxyuracil
  modified_base 6: mod_base = OTHER; note = "t" is deoxyuracil
aagtgtaagc

SEQ ID NO: 168 - DNA, 10 bp, Synthetic construct
  source 1..10: mol_type = other DNA
  modified_base 2: mod_base = OTHER; note = "t" is deoxyuracil
ctacgcacag

SEQ ID NO: 169 - DNA, 10 bp, Synthetic construct
  source 1..10: mol_type = other DNA
  modified_base 1: mod_base = OTHER; note = "t" is deoxyuracil
tgcgccacaa

SEQ ID NO: 170 - DNA, 51 bp, Synthetic construct
  source 1..51: mol_type = other DNA
acagagtggtgagcaattctagccttctcgcatcatacaggttgctcacca

SEQ ID NO: 171 - DNA, 35 bp, Synthetic construct
  source 1..35: mol_type = other DNA
  modified_base 9: mod_base = OTHER; note = "t" is deoxyuracil
  modified_base 12: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagaatcatcgacttctagccttctcgcatca

SEQ ID NO: 172 - DNA, 35 bp, Synthetic construct
  source 1..35: mol_type = other DNA
  modified_base 7: mod_base = OTHER; note = "t" is deoxyuracil
  modified_base 11: mod_base = OTHER; note = "t" is deoxyuracil
  modified_base 16: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagtgactcgcgtttctagccttctcgcatca

SEQ ID NO: 173 - DNA, 35 bp, Synthetic construct
  source 1..35: mol_type = other DNA
  modified_base 7: mod_base = OTHER; note = "t" is deoxyuracil
  modified_base 10: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagttcttcacagttctagccttctcgcatca

SEQ ID NO: 174 - DNA, 35 bp, Synthetic construct
  source 1..35: mol_type = other DNA
  modified_base 10: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagccgttgcgtattctagccttctcgcatca

SEQ ID NO: 175 - DNA, 35 bp, Synthetic construct
  source 1..35: mol_type = other DNA
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagacgcagtggattctagccttctcgcatca

SEQ ID NO: 176 - DNA, 35 bp, Synthetic construct
  source 1..35: mol_type = other DNA
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagccaggctcaattctagccttctcgcatca

SEQ ID NO: 177 - DNA, 35 bp, Synthetic construct
  source 1..35: mol_type = other DNA
  modified_base 10: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagcgattcactattctagccttctcgcatca

SEQ ID NO: 178 - DNA, 35 bp, Synthetic construct
  source 1..35: mol_type = other DNA
  modified_base 10: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagcagtacaggcttctagccttctcgcatca

SEQ ID NO: 179 - DNA, 35 bp, Synthetic construct
  source 1..35: mol_type = other DNA
  modified_base 10: mod_base = OTHER; note = "t" is deoxyuracil
  modified_base 13: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagagcttatcacttctagccttctcgcatca

SEQ ID NO: 180 - DNA, 35 bp, Synthetic construct
  source 1..35: mol_type = other DNA
  modified_base 7: mod_base = OTHER; note = "t" is deoxyuracil
  modified_base 11: mod_base = OTHER; note = "t" is deoxyuracil
  modified_base 14: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagttcgtggtagttctagccttctcgcatca

SEQ ID NO: 181 - DNA, 35 bp, Synthetic construct
  source 1..35: mol_type = other DNA
  modified_base 10: mod_base = OTHER; note = "t" is deoxyuracil
  modified_base 15: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagaacttgccttttctagccttctcgcatca

SEQ ID NO: 182 - DNA, 35 bp, Synthetic construct
  source 1..35: mol_type = other DNA
  modified_base 10: mod_base = OTHER; note = "t" is deoxyuracil
  modified_base 12: mod_base = OTHER; note = "t" is deoxyuracil
  modified_base 20: mod_base = OTHER; note = "t" is deoxyuracil
  modified_base 25: mod_base = OTHER; note = "t" is deoxyuracil
  modified_base 28: mod_base = OTHER; note = "t" is deoxyuracil
  modified_base 33: mod_base = OTHER; note = "t" is deoxyuracil
acagagccatctagaattctagccttctcgcatca

SEQ ID NO: 183 - DNA, 16 bp, Synthetic construct
  source 1..16: mol_type = other DNA
tacagggtcgatgatt

SEQ ID NO: 184 - DNA, 16 bp, Synthetic construct
  source 1..16: mol_type = other DNA
tacaggacgcgagtca

SEQ ID NO: 185 - DNA, 16 bp, Synthetic construct
  source 1..16: mol_type = other DNA
tacaggctgtgaagaa

SEQ ID NO: 186 - DNA, 16 bp, Synthetic construct
  source 1..16: mol_type = other DNA
tacaggtacgcaacgg

SEQ ID NO: 187 - DNA, 16 bp, Synthetic construct
  source 1..16: mol_type = other DNA
tacaggtccactgcgt

SEQ ID NO: 188 - DNA, 16 bp, Synthetic construct
  source 1..16: mol_type = other DNA
tacaggttgagcctgg

SEQ ID NO: 189 - DNA, 16 bp, Synthetic construct
  source 1..16: mol_type = other DNA
tacaggtagtgaatcg

SEQ ID NO: 190 - DNA, 16 bp, Synthetic construct
  source 1..16: mol_type = other DNA
tacagggcctgtactg

SEQ ID NO: 191 - DNA, 16 bp, Synthetic construct
  source 1..16: mol_type = other DNA
tacagggtgataagct

SEQ ID NO: 192 - DNA, 16 bp, Synthetic construct
  source 1..16: mol_type = other DNA
tacaggctaccacgaa

SEQ ID NO: 193 - DNA, 16 bp, Synthetic construct
  source 1..16: mol_type = other DNA
tacaggaaggcaagtt

SEQ ID NO: 194 - DNA, 16 bp, Synthetic construct
  source 1..16: mol_type = other DNA
tacaggttctagatgg

SEQ ID NO: 195 - DNA, 10 bp, Synthetic construct
  source 1..10: mol_type = other DNA
  modified_base 3: mod_base = OTHER; note = "t" is deoxyuracil
  modified_base 6: mod_base = OTHER; note = "t" is deoxyuracil
aatcatcgac

SEQ ID NO: 196 - DNA, 10 bp, Synthetic construct
  source 1..10: mol_type = other DNA
  modified_base 1: mod_base = OTHER; note = "t" is deoxyuracil
  modified_base 5: mod_base = OTHER; note = "t" is deoxyuracil
  modified_base 10: mod_base = OTHER; note = "t" is deoxyuracil
tgactcgcgt

SEQ ID NO: 197 - DNA, 10 bp, Synthetic construct
  source 1..10: mol_type = other DNA
  modified_base 1: mod_base = OTHER; note = "t" is deoxyuracil
  modified_base 4: mod_base = OTHER; note = "t" is deoxyuracil
ttcttcacag

SEQ ID NO: 198 - DNA, 10 bp, Synthetic construct
  source 1..10: mol_type = other DNA
  modified_base 4: mod_base = OTHER; note = "t" is deoxyuracil
  modified_base 9: mod_base = OTHER; note = "t" is deoxyuracil
ccgttgcgta

SEQ ID NO: 199 - DNA, 10 bp, Synthetic construct
  source 1..10: mol_type = other DNA
  modified_base 7: mod_base = OTHER; note = "t" is deoxyuracil
acgcagtgga

SEQ ID NO: 200 - DNA, 10 bp, Synthetic construct
  source 1..10: mol_type = other DNA
  modified_base 7: mod_base = OTHER; note = "t" is deoxyuracil
ccaggctcaa

SEQ ID NO: 201 - DNA, 10 bp, Synthetic construct
  source 1..10: mol_type = other DNA
  modified_base 4: mod_base = OTHER; note = "t" is deoxyuracil
  modified_base 9: mod_base = OTHER; note = "t" is deoxyuracil
cgattcacta

SEQ ID NO: 202 - DNA, 10 bp, Synthetic construct
  source 1..10: mol_type = other DNA
  modified_base 4: mod_base = OTHER; note = "t" is deoxyuracil
cagtacaggc

SEQ ID NO: 203 - DNA, 10 bp, Synthetic construct
  source 1..10: mol_type = other DNA
  modified_base 4: mod_base = OTHER; note = "t" is deoxyuracil
  modified_base 7: mod_base = OTHER; note = "t" is deoxyuracil
agcttatcac

SEQ ID NO: 204 - DNA, 10 bp, Synthetic construct
  source 1..10: mol_type = other DNA
  modified_base 1: mod_base = OTHER; note = "t" is deoxyuracil
  modified_base 5: mod_base = OTHER; note = "t" is deoxyuracil
  modified_base 8: mod_base = OTHER; note = "t" is deoxyuracil
ttcgtggtag

SEQ ID NO: 205 - DNA, 10 bp, Synthetic construct
  source 1..10: mol_type = other DNA
  modified_base 4: mod_base = OTHER; note = "t" is deoxyuracil
  modified_base 9: mod_base = OTHER; note = "t" is deoxyuracil
aacttgcctt

SEQ ID NO: 206 - DNA, 10 bp, Synthetic construct
  source 1..10: mol_type = other DNA
  modified_base 4: mod_base = OTHER; note = "t" is deoxyuracil
  modified_base 6: mod_base = OTHER; note = "t" is deoxyuracil
ccatctagaa

SEQ ID NO: 207 - DNA, 53 bp, Synthetic construct
  source 1..53: mol_type = other DNA
  modified_base 17: mod_base = OTHER; note = "t" is deoxyuracil
agacgaacctctaacatttccctacacgacgctcttccgatcttgttagaggt

SEQ ID NO: 208 - DNA, 53 bp, Synthetic construct
  source 1..53: mol_type = other DNA
  modified_base 17: mod_base = OTHER; note = "t" is deoxyuracil
agacgaaatgaggaactttccctacacgacgctcttccgatctgttcctcatt

SEQ ID NO: 209 - DNA, 53 bp, Synthetic construct
  source 1..53: mol_type = other DNA
  modified_base 17: mod_base = OTHER; note = "t" is deoxyuracil
agacgactaataccagtttccctacacgacgctcttccgatctctggtattag

SEQ ID NO: 210 - DNA, 53 bp, Synthetic construct
  source 1..53: mol_type = other DNA
  modified_base 17: mod_base = OTHER; note = "t" is deoxyuracil
agacgattggaggccttttccctacacgacgctcttccgatctaggcctccaa

SEQ ID NO: 211 - DNA, 53 bp, Synthetic construct
  source 1..53: mol_type = other DNA
  modified_base 17: mod_base = OTHER; note = "t" is deoxyuracil
agacgatcatagccgatttccctacacgacgctcttccgatcttcggctatga

SEQ ID NO: 212 - DNA, 53 bp, Synthetic construct
  source 1..53: mol_type = other DNA
  modified_base 17: mod_base = OTHER; note = "t" is deoxyuracil
agacgatatggctcgctttccctacacgacgctcttccgatctgcgagccata

SEQ ID NO: 213 - DNA, 53 bp, Synthetic construct
  source 1..53: mol_type = other DNA
  modified_base 17: mod_base = OTHER; note = "t" is deoxyuracil
agacgacctatgaaggtttccctacacgacgctcttccgatctccttcatagg

SEQ ID NO: 214 - DNA, 53 bp, Synthetic construct
  source 1..53: mol_type = other DNA
  modified_base 17: mod_base = OTHER; note = "t" is deoxyuracil
agacgacgtctcaagctttccctacacgacgctcttccgatctgcttgagacg

SEQ ID NO: 215 - DNA, 53 bp, Synthetic construct
  source 1..53: mol_type = other DNA
  modified_base 17: mod_base = OTHER; note = "t" is deoxyuracil
agacgacatactcctctttccctacacgacgctcttccgatctgaggagtatg

SEQ ID NO: 216 - DNA, 53 bp, Synthetic construct
  source 1..53: mol_type = other DNA
  modified_base 17: mod_base = OTHER; note = "t" is deoxyuracil
agacgaaagtgtaagctttccctacacgacgctcttccgatctgcttacactt

SEQ ID NO: 217 - DNA, 53 bp, Synthetic construct
  source 1..53: mol_type = other DNA
  modified_base 17: mod_base = OTHER; note = "t" is deoxyuracil
agacgactacgcacagtttccctacacgacgctcttccgatctctgtgcgtag

SEQ ID NO: 218 - DNA, 53 bp, Synthetic construct
  source 1..53: mol_type = other DNA
  modified_base 17: mod_base = OTHER; note = "t" is deoxyuracil
agacgatgcgccacaatttccctacacgacgctcttccgatctttgtggcgca

SEQ ID NO: 219 - DNA, 10 bp, Synthetic construct
  source 1..10: mol_type = other DNA
acctctaaca

SEQ ID NO: 220 - DNA, 10 bp, Synthetic construct
  source 1..10: mol_type = other DNA
aatgaggaac

SEQ ID NO: 221 - DNA, 10 bp, Synthetic construct
  source 1..10: mol_type = other DNA
ctaataccag

SEQ ID NO: 222 - DNA, 10 bp, Synthetic construct
  source 1..10: mol_type = other DNA
ttggaggcct

SEQ ID NO: 223 - DNA, 10 bp, Synthetic construct
  source 1..10: mol_type = other DNA
tcatagccga

SEQ ID NO: 224 - DNA, 10 bp, Synthetic construct
  source 1..10: mol_type = other DNA
tatggctcgc

SEQ ID NO: 225 - DNA, 10 bp, Synthetic construct
  source 1..10: mol_type = other DNA
cctatgaagg

SEQ ID NO: 226 - DNA, 10 bp, Synthetic construct
  source 1..10: mol_type = other DNA
cgtctcaagc

SEQ ID NO: 227 - DNA, 10 bp, Synthetic construct
  source 1..10: mol_type = other DNA
catactcctc

SEQ ID NO: 228 - DNA, 10 bp, Synthetic construct
  source 1..10: mol_type = other DNA
aagtgtaagc

SEQ ID NO: 229 - DNA, 10 bp, Synthetic construct
  source 1..10: mol_type = other DNA
ctacgcacag

SEQ ID NO: 230 - DNA, 10 bp, Synthetic construct
  source 1..10: mol_type = other DNA
tgcgccacaa

SEQ ID NO: 231 - DNA, 53 bp, Synthetic construct
  source 1..53: mol_type = other DNA
  modified_base 17: mod_base = OTHER; note = "t" is deoxyuracil
acagagaatcatcgactttccctacacgacgctcttccgatctgtcgatgatt

SEQ ID NO: 232 - DNA, 53 bp, Synthetic construct
  source 1..53: mol_type = other DNA
  modified_base 17: mod_base = OTHER; note = "t" is deoxyuracil
acagagtgactcgcgttttccctacacgacgctcttccgatctacgcgagtca

SEQ ID NO: 233 - DNA, 53 bp, Synthetic construct
  source 1..53: mol_type = other DNA
  modified_base 17: mod_base = OTHER; note = "t" is deoxyuracil
acagagttcttcacagtttccctacacgacgctcttccgatctctgtgaagaa

SEQ ID NO: 234 - DNA, 53 bp, Synthetic construct
  source 1..53: mol_type = other DNA
  modified_base 17: mod_base = OTHER; note = "t" is deoxyuracil
acagagccgttgcgtatttccctacacgacgctcttccgatcttacgcaacgg

SEQ ID NO: 235 - DNA, 53 bp, Synthetic construct
  source 1..53: mol_type = other DNA
  modified_base 17: mod_base = OTHER; note = "t" is deoxyuracil
acagagacgcagtggatttccctacacgacgctcttccgatcttccactgcgt

SEQ ID NO: 236 - DNA, 53 bp, Synthetic construct
  source 1..53: mol_type = other DNA
  modified_base 17: mod_base = OTHER; note = "t" is deoxyuracil
acagagccaggctcaatttccctacacgacgctcttccgatctttgagcctgg

SEQ ID NO: 237 - DNA, 53 bp, Synthetic construct
  source 1..53: mol_type = other DNA
  modified_base 17: mod_base = OTHER; note = "t" is deoxyuracil
acagagcgattcactatttccctacacgacgctcttccgatcttagtgaatcg

SEQ ID NO: 238 - DNA, 53 bp, Synthetic construct
  source 1..53: mol_type = other DNA
  modified_base 17: mod_base = OTHER; note = "t" is deoxyuracil
acagagcagtacaggctttccctacacgacgctcttccgatctgcctgtactg

SEQ ID NO: 239 - DNA, 53 bp, Synthetic construct
  source 1..53: mol_type = other DNA
  modified_base 17: mod_base = OTHER; note = "t" is deoxyuracil
acagagagcttatcactttccctacacgacgctcttccgatctgtgataagct

SEQ ID NO: 240 - DNA, 53 bp, Synthetic construct
  source 1..53: mol_type = other DNA
  modified_base 17: mod_base = OTHER; note = "t" is deoxyuracil
acagagttcgtggtagtttccctacacgacgctcttccgatctctaccacgaa

SEQ ID NO: 241 - DNA, 53 bp, Synthetic construct
  source 1..53: mol_type = other DNA
  modified_base 17: mod_base = OTHER; note = "t" is deoxyuracil
acagagaacttgcctttttccctacacgacgctcttccgatctaaggcaagtt

SEQ ID NO: 242 - DNA, 53 bp, Synthetic construct
  source 1..53: mol_type = other DNA
  modified_base 17: mod_base = OTHER; note = "t" is deoxyuracil
acagagccatctagaatttccctacacgacgctcttccgatctttctagatgg

SEQ ID NO: 243 - DNA, 10 bp, Synthetic construct
  source 1..10: mol_type = other DNA
aatcatcgac

SEQ ID NO: 244 - DNA, 10 bp, Synthetic construct
  source 1..10: mol_type = other DNA
tgactcgcgt

SEQ ID NO: 245 - DNA, 10 bp, Synthetic construct
  source 1..10: mol_type = other DNA
ttcttcacag

SEQ ID NO: 246 - DNA, 10 bp, Synthetic construct
  source 1..10: mol_type = other DNA
ccgttgcgta

SEQ ID NO: 247 - DNA, 10 bp, Synthetic construct
  source 1..10: mol_type = other DNA
acgcagtgga

SEQ ID NO: 248 - DNA, 10 bp, Synthetic construct
  source 1..10: mol_type = other DNA
ccaggctcaa

SEQ ID NO: 249 - DNA, 10 bp, Synthetic construct
  source 1..10: mol_type = other DNA
cgattcacta

SEQ ID NO: 250 - DNA, 10 bp, Synthetic construct
  source 1..10: mol_type = other DNA
cagtacaggc

SEQ ID NO: 251 - DNA, 10 bp, Synthetic construct
  source 1..10: mol_type = other DNA
agcttatcac

SEQ ID NO: 252 - DNA, 10 bp, Synthetic construct
  source 1..10: mol_type = other DNA
ttcgtggtag

SEQ ID NO: 253 - DNA, 10 bp, Synthetic construct
  source 1..10: mol_type = other DNA
aacttgcctt

SEQ ID NO: 254 - DNA, 10 bp, Synthetic construct
  source 1..10: mol_type = other DNA
ccatctagaa

SEQ ID NO: 255 - DNA, 20 bp, Synthetic construct
  source 1..20: mol_type = other DNA
agaatagtaaccgacagtgc

SEQ ID NO: 256 - DNA, 20 bp, Synthetic construct
  source 1..20: mol_type = other DNA
agaaaattaagcggcagtgc

SEQ ID NO: 257 - DNA, 20 bp, Synthetic construct
  source 1..20: mol_type = other DNA
agaaagacgaggtccagtgc

SEQ ID NO: 258 - DNA, 20 bp, Synthetic construct
  source 1..20: mol_type = other DNA
agaactaataggctcagtgc

SEQ ID NO: 259 - DNA, 20 bp, Synthetic construct
  source 1..20: mol_type = other DNA
agaagcctaccgatcagtgc

SEQ ID NO: 260 - DNA, 20 bp, Synthetic construct
  source 1..20: mol_type = other DNA
agaacttatcaggtcagtgc

SEQ ID NO: 261 - DNA, 20 bp, Synthetic construct
  source 1..20: mol_type = other DNA
agaatgcatataggcagtgc

SEQ ID NO: 262 - DNA, 20 bp, Synthetic construct
  source 1..20: mol_type = other DNA
agaagcacctgatccagtgc

SEQ ID NO: 263 - DNA, 20 bp, Synthetic construct
  source 1..20: mol_type = other DNA
agaagtactcgtgacagtgc

SEQ ID NO: 264 - DNA, 20 bp, Synthetic construct
  source 1..20: mol_type = other DNA
agaacacatatgcacagtgc

SEQ ID NO: 265 - DNA, 20 bp, Synthetic construct
  source 1..20: mol_type = other DNA
agaacgtactataccagtgc

SEQ ID NO: 266 - DNA, 20 bp, Synthetic construct
  source 1..20: mol_type = other DNA
agaaagtgttgtctcagtgc

SEQ ID NO: 267 - DNA, 20 bp, Synthetic construct
  source 1..20: mol_type = other DNA
agaacggttgcctacagtgc

SEQ ID NO: 268 - DNA, 20 bp, Synthetic construct
  source 1..20: mol_type = other DNA
agaaaccactgttaacatcg

SEQ ID NO: 269 - DNA, 20 bp, Synthetic construct
  source 1..20: mol_type = other DNA
agaatatcagccaaacatcg

SEQ ID NO: 270 - DNA, 20 bp, Synthetic construct
  source 1..20: mol_type = other DNA
agaaccaagccatcacatcg

SEQ ID NO: 271 - DNA, 20 bp, Synthetic construct
  source 1..20: mol_type = other DNA
agaagatcgcctcaacatcg

SEQ ID NO: 272 - DNA, 20 bp, Synthetic construct
  source 1..20: mol_type = other DNA
agaagctctgaaggacatcg

SEQ ID NO: 273 - DNA, 20 bp, Synthetic construct
  source 1..20: mol_type = other DNA
agaacttggcctctacatcg

SEQ ID NO: 274 - DNA, 20 bp, Synthetic construct
  source 1..20: mol_type = other DNA
agaatcatcggaatacatcg

SEQ ID NO: 275 - DNA, 20 bp, Synthetic construct
  source 1..20: mol_type = other DNA
agaatcaccgtataacatcg

SEQ ID NO: 276 - DNA, 20 bp, Synthetic construct
  source 1..20: mol_type = other DNA
agaatgttacctcaacatcg

SEQ ID NO: 277 - DNA, 20 bp, Synthetic construct
  source 1..20: mol_type = other DNA
agaagaaggcctaaacatcg

SEQ ID NO: 278 - DNA, 20 bp, Synthetic construct
  source 1..20: mol_type = other DNA
agaatacgaatcgaacatcg

SEQ ID NO: 279 - DNA, 20 bp, Synthetic construct
  source 1..20: mol_type = other DNA
agaacgaatggtaaacatcg

SEQ ID NO: 280 - DNA, 20 bp, Synthetic construct
  source 1..20: mol_type = other DNA
agaaccgcaataggacatcg

SEQ ID NO: 281 - DNA, 20 bp, Synthetic construct
  source 1..20: mol_type = other DNA
agaagcaccgctattcgtct

SEQ ID NO: 282 - DNA, 20 bp, Synthetic construct
  source 1..20: mol_type = other DNA
agaatgttagaggttcgtct

SEQ ID NO: 283 - DNA, 20 bp, Synthetic construct
  source 1..20: mol_type = other DNA
agaagttcctcatttcgtct

SEQ ID NO: 284 - DNA, 20 bp, Synthetic construct
  source 1..20: mol_type = other DNA
agaactggtattagtcgtct

SEQ ID NO: 285 - DNA, 20 bp, Synthetic construct
  source 1..20: mol_type = other DNA
agaaaggcctccaatcgtct

SEQ ID NO: 286 - DNA, 20 bp, Synthetic construct
  source 1..20: mol_type = other DNA
agaatcggctatgatcgtct

SEQ ID NO: 287 - DNA, 20 bp, Synthetic construct
  source 1..20: mol_type = other DNA
agaagcgagccatatcgtct

SEQ ID NO: 288 - DNA, 20 bp, Synthetic construct
  source 1..20: mol_type = other DNA
agaaccttcataggtcgtct

SEQ ID NO: 289 - DNA, 20 bp, Synthetic construct
  source 1..20: mol_type = other DNA
agaagcttgagacgtcgtct

SEQ ID NO: 290 - DNA, 20 bp, Synthetic construct
  source 1..20: mol_type = other DNA
agaagaggagtatgtcgtct

SEQ ID NO: 291 - DNA, 20 bp, Synthetic construct
  source 1..20: mol_type = other DNA
agaagcttacactttcgtct

SEQ ID NO: 292 - DNA, 20 bp, Synthetic construct
  source 1..20: mol_type = other DNA
agaactgtgcgtagtcgtct

SEQ ID NO: 293 - DNA, 20 bp, Synthetic construct
  source 1..20: mol_type = other DNA
agaattgtggcgcatcgtct

SEQ ID NO: 294 - DNA, 20 bp, Synthetic construct
  source 1..20: mol_type = other DNA
agaattgctcaccactctgt

SEQ ID NO: 295 - DNA, 20 bp, Synthetic construct
  source 1..20: mol_type = other DNA
agaagtcgatgattctctgt

SEQ ID NO: 296 - DNA, 20 bp, Synthetic construct
  source 1..20: mol_type = other DNA
agaaacgcgagtcactctgt

SEQ ID NO: 297 - DNA, 20 bp, Synthetic construct
  source 1..20: mol_type = other DNA
agaactgtgaagaactctgt

SEQ ID NO: 298 - DNA, 20 bp, Synthetic construct
  source 1..20: mol_type = other DNA
agaatacgcaacggctctgt

SEQ ID NO: 299 - DNA, 20 bp, Synthetic construct
  source 1..20: mol_type = other DNA
agaatccactgcgtctctgt

SEQ ID NO: 300 - DNA, 20 bp, Synthetic construct
  source 1..20: mol_type = other DNA
agaattgagcctggctctgt

SEQ ID NO: 301 - DNA, 20 bp, Synthetic construct
  source 1..20: mol_type = other DNA
agaatagtgaatcgctctgt

SEQ ID NO: 302 - DNA, 20 bp, Synthetic construct
  source 1..20: mol_type = other DNA
agaagcctgtactgctctgt

SEQ ID NO: 303 - DNA, 20 bp, Synthetic construct
  source 1..20: mol_type = other DNA
agaagtgataagctctctgt

SEQ ID NO: 304 - DNA, 20 bp, Synthetic construct
  source 1..20: mol_type = other DNA
agaactaccacgaactctgt

SEQ ID NO: 305 - DNA, 20 bp, Synthetic construct
  source 1..20: mol_type = other DNA
agaaaaggcaagttctctgt

SEQ ID NO: 306 - DNA, 20 bp, Synthetic construct
  source 1..20: mol_type = other DNA
agaattctagatggctctgt

SEQ ID NO: 307 - DNA, 54 bp, Synthetic construct
  source 1..54: mol_type = other DNA
  modified_base 17: mod_base = OTHER; note = "t" is deoxyuracil
  modified_base 24: mod_base = OTHER; note = "t" is deoxyuracil
  modified_base 30: mod_base = OTHER; note = "t" is deoxyuracil
  modified_base 34: mod_base = OTHER; note = "t" is deoxyuracil
  modified_base 39: mod_base = OTHER; note = "t" is deoxyuracil
  modified_base 44: mod_base = OTHER; note = "t" is deoxyuracil
  modified_base 47: mod_base = OTHER; note = "t" is deoxyuracil
  modified_base 52: mod_base = OTHER; note = "t" is deoxyuracil
ctgtctcttatacacatctgcactgccgcttaattttctagccttctcgcatca

SEQ ID NO: 308 - DNA, 41 bp, Synthetic construct
  source 1..41: mol_type = other DNA
acatcgaattaagcggcagtgcagatgtgtataagagacag

SEQ ID NO: 309 - DNA, 57 bp, Synthetic construct
  source 1..57: mol_type = other DNA
tcgtcttatcagccaaacatcgaattaagcggcagtgcagatgtgtataagagacag

SEQ ID NO: 310 - DNA, 73 bp, Synthetic construct
  source 1..73: mol_type = other DNA
tatcagccaaacatcgagacgaggtccagtgcagatgtgtataagagacagcttctacttccactaccatgag

SEQ ID NO: 311 - DNA, 73 bp, Synthetic construct
  source 1..73: mol_type = other DNA
tatcagccaaacatcgagacgaggtccagtgcagatgtgtataagagacaggcttaggaggcataaacaggtg

SEQ ID NO: 312 - DNA, 22 bp, Synthetic construct
  source 1..22: mol_type = other DNA
cttctacttccactaccatgag

SEQ ID NO: 313 - DNA, 22 bp, Synthetic construct
  source 1..22: mol_type = other DNA
gcttaggaggcataaacaggtg

(19) United States
(12) Patent Application Publication
(10) Pub. No.: US 2026/0258398 A1
(43) Pub. Date: Sep. 3, 2026

(54) Methods and Compositions for Generating Spatially Resolved Genomic Profiles From Tissues

(71) Applicant: The Regents of the University of California, Oakland, CA (US)

(72) Inventor: Chongyuan Luo, Los Angeles, CA (US)

(21) Appl. No.: 18/867,516

(22) PCT Filed: May 22, 2023
(86) PCT No.: PCT/US2023/023144

§ 371 Date: Nov. 20, 2024

Related U.S. Application Data
(60) Provisional application No. 63/346,977, filed on May 30, 2022.

(51) Int. Cl. C12N 15/10

(52) U.S. Cl. CPC C12N 15/1065

(57) ABSTRACT

Disclosed herein are methods and compositions for tagging nucleic acid molecules of interest with spatial information about the cells from which the nucleic acid molecules were derived from were located in a tissue sample or an array of cells.

### CROSS-REFERENCE TO RELATED APPLICATIONS

This application claims the benefit of U.S. Patent Application No. 63/346,977, filed May 30, 2022, which is herein incorporated by reference in its entirety.

### REFERENCE TO A SEQUENCE LISTING SUBMITTED VIA PATENTCENTER

The content of the XML file of the sequence listing named “20230522_034044_237WO1 ST26” which is 526,434 bytes in size was created on May 22, 2023 and electronically submitted via PatentCenter herewith the application is incorporated herein by reference in its entirety.

### BACKGROUND OF THE INVENTION

### 1. Field of the Invention

The field generally relates to transcriptomics and high-throughput sequencing.

### 2. Description of the Related Art

Transcriptomics methods in the art are useful tools that allow the analysis of RNA transcripts in cells and tissues of organisms. The primary methods employed today may be generally divided into two types: (1) microarray-based methods, which assay a set of predetermined sequences, and (2) RNA-Seq, which uses high-throughput sequencing to assay all transcripts in a given sample.

Spatial transcriptomic methods characterize the transcriptomes of cells according to their location in a tissue sample, e.g., a histological tissue section. Numerous spatial transcriptomic methods have been developed and can be generally divided into the following five categories: microdissection methods, fluorescent in situ hybridization methods, in situ sequencing methods, in situ capture methods, and in silico methods.

Unfortunately, spatial transcriptomic methods employed in the art today suffer various problems, e.g., poor resolution at the single-cell level, require highly specialized equipment such as microfluidic controllers, inability to select cells or regions of interests for analysis, and limited to relatively small areas of tissue.

### SUMMARY OF THE INVENTION

Provided herein are Ligation Adaptors for tagging a nucleic acid molecule with a barcode sequence, the Ligation Adaptors comprise a ligation adaptor sequence of about 6-10 bases long with a photocleavable linker (PC Linker) having a phosphate group that is linked to one end of the ligation adaptor sequence, and the barcode sequence linked to the other end of the ligation adaptor sequence. In some embodiments, the Ligation Adaptors comprise, from the 5′ to 3′ end, a sequence that is the reverse complement of the ligation adaptor sequence which is linked to a sequence that is the reverse complement of the barcode sequence which is linked to a hairpin loop sequence of about 15-25 bases long which is linked to the PC Linker which is linked to the ligation adaptor sequence which is linked to the barcode sequence, and preferably the barcode sequence is selected from the group consisting of SEQ ID NO: 1 to SEQ ID NO: 52, more preferably the barcode sequence is selected from the group consisting of SEQ ID NO: 1 to SEQ ID NO: 48. In some embodiments, the barcode sequence is 8-12 bases, 9-11 bases, or 10 bases long, and optionally the barcode sequence is non-naturally occurring in the genome from which the nucleic acid molecule was obtained. In some embodiments, the barcode sequence is selected from the group consisting of SEQ ID NO: 1 to SEQ ID NO: 52, and complementary and reverse complementary sequences thereof; and optionally the barcode sequence is non-naturally occurring in the genome from which the nucleic acid molecule was obtained. In some embodiments, the barcode sequence is selected from the group consisting of SEQ ID NO: 1 to SEQ ID NO: 48, and complementary and reverse complementary sequences thereof, and optionally the barcode sequence is non-naturally occurring in the genome from which the nucleic acid molecule was obtained. In some embodiments, the Ligation Adaptor comprises a hairpin loop sequence of about 15-25 bases long that has one end linked to the PC Linker. In some embodiments, the Ligation Adaptor comprises a sequence that is a reverse complement of the barcode sequence, said sequence attached to the end of the hairpin loop sequence opposite to the end attached to the PC Linker. In some embodiments, the hairpin loop sequence is TTCUAGCCUTCUCGCAUCA (SEQ ID NO: 53). In some embodiments, the nucleic acid molecule to be tagged comprises a photocleavable blocker (“PC Blocker”) linked to an initial ligation adaptor sequence. In some embodiments, the Ligation Adaptor comprises a sequence that is a reverse complement of an initial ligation adaptor sequence present on the nucleic acid molecule to be tagged. In some embodiments, the ligation adaptor sequence and the initial ligation adaptor sequence are each independently selected from the group consisting of CAGTGC, GCACUG, CGAUGU, AGACGA, ACAGAG, and reverse complements thereof. In some embodiments, the sequence upstream of the PC Blocker is selected from the group consisting of SEQ ID NO: 60 to SEQ ID NO: 71; SEQ ID NO: 97 to SEQ ID NO: 108; SEQ ID NO: 134 to SEQ ID NO: 145; and SEQ ID NO: 171 to SEQ ID NO: 182 and the sequence downstream of the PC Blocker is selected from the group consisting of SEQ ID NO: 72 to SEQ ID NO: 83; SEQ ID NO: 109 to SEQ ID NO: 120; SEQ ID NO: 146 to SEQ ID NO: 157; and SEQ ID NO: 183 to SEQ ID NO: 194. In some embodiments, the Ligation Adaptor is selected from the group consisting of S1-1 to S1-12, S2-1 to S2-12, S3-1 to S3-12, and S4-1 to S4-12 Ligation Adaptors.

Also provided are methods of tagging a nucleic acid molecule with a barcode sequence using a Ligation Adaptor as described herein, wherein the barcode sequence provides information about the location of a cell in a tissue sample or in an array of cells to which the nucleic acid molecule was derived, said method comprises performing the following steps (a) cleaving the PC Blocker by selectively exposing the cell to light; (b) adding the Ligation Adaptor (“first Ligation Adaptor”) to the nucleic acid molecule (“first nucleic acid molecule”) within the cell (“first cell”); and (c) adding ligase to the cell; and (d) optionally, repeating steps (a)-(c) one or more times with a subsequent Ligation Adaptor (“first subsequent Ligation Adaptor”), wherein each subsequent Ligation Adaptor comprises a sequence that is the reverse complement of the ligation adaptor sequence of the preceding Ligation Adaptor, and the barcode sequence of each subsequent Ligation Adaptor may be the same or different from the barcode sequence of the preceding Ligation Adaptor; while the cell is intact and remains a part of the tissue sample or the array. In some embodiments, selectively exposing the cell to light comprises using a photomask to block other cells in the tissue sample or the array from exposure to light and/or using a laser or a microscope such as an epifluorescence microscope, a one-photon laser scanning microscope, or a two-photon scanning microscope to focus light on the cell. In some embodiments, the methods further comprise tagging a second nucleic acid molecule of a second cell, which comprises (e) performing steps (a)-(c) with a second Ligation Adaptor having a second barcode sequence that is different from the barcode sequence of the first Ligation Adaptor, and (f) optionally repeating steps (a)-(c) one or more times with a second subsequent Ligation Adaptor, wherein each second subsequent Ligation Adaptor comprises a sequence that is the reverse complement of the ligation adaptor sequence of the preceding second Ligation Adaptor, and the barcode sequence of each second subsequent Ligation Adaptor may be the same or different from the barcode sequence of the preceding second Ligation Adaptor; while the second cell is intact and remains a part of the tissue sample or the array. In the methods described herein, (i) the barcode sequences of the first Ligation Adaptor and the first subsequent Ligation Adaptor(s) are different, (ii) the barcode sequences of the second Ligation Adaptor and the second subsequent Ligation Adaptor(s) are different, or both (i) and (ii). In the methods described herein, the PC Linkers of the Ligation Adaptor and the PC Blocker are the same or different. In some embodiments, the methods further comprise providing a Transposase Recognition Sequence, e.g., SEQ ID NO: 56, downstream of the initial ligation adaptor sequence. In some embodiments, the nucleic acid molecules of a cell or cells in different sections of the tissue sample or the array are tagged with unique barcode sequences and/or unique combinations of barcode sequences. In some embodiments, the methods further comprise obtaining an extract of all the nucleotide molecules of the cells of the tissue sample or the array after tagging the first and/or second nucleic acid molecules with one or more barcode sequences, and sequencing the nucleic acid molecules having the one or more barcode sequences. In some embodiments, the methods further comprise identifying the barcode sequence(s), number of barcode sequences, and/or combination of different barcode sequences ligated to a given nucleic acid molecule and correlating such to the position of the cell in the tissue sample or array that was treated with the particular Ligation Adaptor(s) that would necessarily result in the identified barcode sequence(s), number of barcode sequences, and/or combination of different barcode sequences ligated to the given nucleic acid molecule.

Also provided are nucleic acid molecules comprising (i) a barcode sequence selected from the group consisting of SEQ ID NO: 1 to SEQ ID NO: 52, and complementary and reverse complementary sequences thereof; (ii) linked to a universal sequencing adaptor and/or a ligation adaptor sequence. In some embodiments, the nucleic acid molecule further comprises a sequence that is the reverse complement of the barcode sequence. In some embodiments, the nucleic acid molecule contains a uracil base preceding the universal sequencing adaptor, both of which are flanked by the barcode sequence and the reverse complement of the barcode sequence. In some embodiments, the ligation adaptor sequence is selected from the group consisting of CAGTGC, GCACUG, CGAUGU, AGACGA, ACAGAG, and reverse complements thereof. In some embodiments, the universal sequencing adaptor sequence is TTCCCTACACGACGCTCTTCCGATCT (SEQ ID NO: 54). In some embodiments, the nucleic acid molecule comprises or consists of a sequence selected from the group consisting of: SEQ ID NO: 60 to SEQ ID NO: 71; SEQ ID NO: 97 to SEQ ID NO: 108; SEQ ID NO: 134 to SEQ ID NO: 145; and SEQ ID NO: 171 to SEQ ID NO: 182. In some embodiments, the nucleic acid molecule comprises or consists of a sequence selected from the group consisting of: SEQ ID NO: 72 to SEQ ID NO: 83; SEQ ID NO: 109 to SEQ ID NO: 120; SEQ ID NO: 146 to SEQ ID NO: 157; and SEQ ID NO: 183 to SEQ ID NO: 194. In some embodiments, the nucleic acid molecule comprises or consists of a sequence selected from the group consisting of: SEQ ID NO: 207 to SEQ ID NO: 218; and SEQ ID NO: 231 to SEQ ID NO: 242. In some embodiments, the nucleic acid molecule comprises or consists of a sequence selected from the group consisting of: SEQ ID NO: 255 to SEQ ID NO: 306.

Also provided are kits comprising a plurality of Ligation Adaptors as described herein packaged together. In some embodiments, the kits comprises one or more Ligation Adaptors as described herein packaged together with one or more nucleic acid molecules comprising a barcode sequence selected from the group consisting of SEQ ID NO: 1 to SEQ ID NO: 52, and complementary and reverse complementary sequences thereof; linked to a universal sequencing adaptor and/or a ligation adaptor sequence. In some embodiments, the nucleic acid molecule further comprises a sequence that is the reverse complement of the barcode sequence. In some embodiments, the nucleic acid molecule contains a uracil base preceding the universal sequencing adaptor, both of which are flanked by the barcode sequence and the reverse complement of the barcode sequence. In some embodiments, the ligation adaptor sequence is selected from the group consisting of CAGTGC, GCACUG, CGAUGU, AGACGA, ACAGAG, and reverse complements thereof. In some embodiments, the universal sequencing adaptor sequence is TTCCCTACACGACGCTCTTCCGATCT (SEQ ID NO: 54). In some embodiments, the kits comprise one or more Ligation Adaptors selected from the group consisting of S1-1 to S1-12, S2-1 to S2-12, S3-1 to S3-12, and S4-1 to S4-12 Ligation Adaptors; one or more sequencing adaptors selected from the group consisting of SEQ ID NO: 207 to SEQ ID NO: 218 and SEQ ID NO: 231 to SEQ ID NO: 242; and one or more adaptor blockers selected from the group consisting of: SEQ ID NO: 255 to SEQ ID NO: 306. In some embodiments, the kits further include a pi-Blocker, e.g., SEQ ID NO: 58. In some embodiments, the kits further include one or more sequences selected from SEQ ID NO: 59, SEQ ID NO: 96, SEQ ID NO: 133, and SEQ ID NO: 170. In some embodiments, the kits further include one or more buffer solutions, a DNA ligase, and/or Tn5 transposase.

Also provided are compositions comprising (a) a mixture of one or more Ligation Adaptors as described herein, or (b) a mixture of one or more nucleic acid molecules comprising a barcode sequence selected from the group consisting of SEQ ID NO: 1 to SEQ ID NO: 52, and complementary and reverse complementary sequences thereof; linked to a universal sequencing adaptor and/or a ligation adaptor sequence. In some embodiments, the one or more nucleic acid molecules further comprise a sequence that is the reverse complement of the barcode sequence. In some embodiments, the one or more nucleic acid molecules contain a uracil base preceding the universal sequencing adaptor, both of which are flanked by the barcode sequence and the reverse complement of the barcode sequence. In some embodiments, the ligation adaptor sequence is selected from the group consisting of CAGTGC, GCACUG, CGAUGU, AGACGA, ACAGAG, and reverse complements thereof. In some embodiments, the universal sequencing adaptor sequence is TTCCCTACACGACGCTCTTCCGATCT (SEQ ID NO: 54).

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 schematically shows two ligation-cleavage cycles using the S1-1 and S2-1 Ligation Adaptors exemplified herein, wherein “iSpPC” is representative of a PC Linker. S2-1 Barcode is SEQ ID NO: 13, S1-1 Barcode is SEQ ID NO: 1, Tn5 mosaic end sequence is SEQ ID NO: 56

FIG. 2 schematically shows a 10×10 array of a tissue sample subjected to a first round of ligation-cleavage (indicated as the first number, i.e., “1” preceding the dash) wherein the nucleic acid molecules of the cells in the sections of each column are ligated to a Ligation Adaptor having a unique barcode sequence (the number indicated after the dash selected from 10 different barcode sequences) selected from a set of first Ligation Adaptors having different barcode sequences. That is, all Ligation Adaptors beginning with “1-” are members of the first set of Ligation Adaptors.

FIG. 3 schematically shows the 10×10 array of FIG. 2 being subjected to a second round of ligation-cleavage (indicated as the first number, i.e., “2” preceding the dash) wherein the nucleic acid molecules of the cells in the sections of each row are ligated to a Ligation Adaptor having a unique barcode sequence (the number indicated after the dash selected from 10 different barcode sequences) selected from a set of first Ligation Adaptors having different barcode sequences. That is, all Ligation Adaptors beginning with “2-” are members of the second set of Ligation Adaptors.

FIG. 4 schematically shows the barcode sequences that the nucleic acid molecules derived from cells located in the given section of the 10×10 array will have. The top number in each section is the barcode from the first cleavage-ligation cycle, which will be located upstream of the given Transposase Recognition Sequence. The bottom number in each section is the barcode from the second cleavage-ligation cycle, which will be located upstream of the first barcode. As an example, the Spatial Barcode of the shaded section in the top row is 5′-Barcode 1-Barcode 1-3′, and the Spatial Barcode of the shaded section in the third row is 5′-Barcode 3-Barcode 5-3′.

FIG. 5 schematically shows the Spatial Barcodes that will result when the third row is either blocked from being illuminated with light or the cells in the sections of the third row were not treated with a Ligation Adaptor during the second cleavage-ligation cycle.

FIG. 6 schematically shows an overview of the pi-seq methodology. (A) In pi-ATAC-seq, thin tissue sections are treated with a transposase to insert sequencing adaptors into open chromatin regions. In pi-mC-seq, tissue sections will be treated with bisulfite conversion followed by in situ generation of DNA methylome library. In pi-RNA-seq, in situ reverse transcription will be performed on tissue sections to generated RNA-seq library. (B) Pi-seq tags nucleic acid molecules of cells with unique Spatial Barcodes.

FIG. 7 schematically shows pi-seq sequential ligation reactions resulting in a unique series of barcode sequences that formulate a Spatial Barcode. (A-C) Adaptor designs for pi-ATAC-seq (A), pi-mC-seq (B), and pi-RNA-seq (C). (D) Schematics of the sequential ligation strategy.

FIG. 8-FIG. 11 are photos of in situ regional and single-cell pi-ATAC-seq experiments in mouse brain slices. (FIG. 8) in situ Tn5 transposition (ATAC-seq) in 10 μm mouse brain slice and (FIG. 9) Regional spatial indexing with sequential ligation reaction (in reverse color for improved reproducibility). FIG. 10 shows single-cell resolution spatial indexing using laser scanning microscopy (in reverse color for improved reproducibility). FIG. 11 shows the selective indexing of a subset of single cells using laser scanning microscopy (in reverse color for improved reproducibility).

FIG. 12. Sequencing of pi-ATAC-seq library. (A) Structure of a pi-ATAC-seq reads generated by two cycles of sequential barcode ligation. The top sequence is SEQ ID NO: 310 and the bottom sequence is SEQ ID NO: 311. Stage 2 Barcode is SEQ ID NO: 13, Stage 1 Barcode is SEQ ID NO: 2, Tn5 mosaic end sequence is SEQ ID NO: 56, Genomic Sequence (top) is SEQ ID NO: 312, and Genomic Sequence (bottom) is SEQ ID NO: 313. (B) Sequential ligation of pi-seq Cycle 2 adaptor is strictly dependent on UV deprotection and the previous ligation of Cycle 1 adaptor.

FIG. 13-FIG. 15. Spatial chromatin accessibility profiling of primary prostate tumor tissue. (FIG. 13) Stitched image epifluorescence image of the prostate tumor tissue section. (FIG. 14) Open chromatin peaks identified from the spatial ATAC-seq data. (FIG. 15) Browser views of spatial ATAC-seq signal at MYC and NKX3-1 loci.

### DETAILED DESCRIPTION OF THE INVENTION

Disclosed herein is a spatial “barcode” tagging method that allows one to tag a nucleic acid molecule with information about the particular single cell from which it was derived. The inventive methods are generically referred herein to as “pi-seq” methods because the method involves photonic indexing followed by sequencing to insert one or more “barcodes”, which are unique nucleic acid sequences, in the nucleic acid molecules of a cell or cells at a desired point in or section of a tissue sample (e.g., a histological tissue sample) or an array of cells (e.g., cell clones in a petri-dish) prior to homogenizing the cell(s) to extract the nucleic acid molecules therein for sequencing. Thus, a given nucleic acid molecule having a unique barcode sequence or a unique sequence of different barcodes that were ligated to the nucleic acid molecules when present in the intact cells of the tissue sample or array indicates the location in the tissue sample or array from which the given nucleic acid molecule was derived. Such barcodes that are ligated to nucleic acid molecules present in intact cells of a tissue sample or array of cells are referred to herein as “Spatial Barcodes”.

The Spatial Barcodes are inserted into the nucleic acid molecules of given cells at desired locations of a tissue sample or array of cells using ligation-cleavage reactions controlled by light, e.g., UV light. Specifically, the pi-seq methods described herein employ adaptor tagging of target nucleic acid molecules, e.g., genomic DNA in open chromatin regions, using 5′-ligation adaptors that have a photocleavable structure which, when intact, prevents a nucleic acid sequence from hybridization and ligation thereto. The nucleic acid molecules of specific cells of interest that are to be tagged with (i.e., ligated to) a given barcode are illuminated with light using, e.g., an epifluorescence microscope, one-photon laser scanning microscope, or a two-photon scanning microscope, to cleave the structure and thereby present the 5′ end of ligation adaptor sequence with a phosphate group thereon so that it can hybridize with its reverse-complement. In some embodiments, the photon laser scanning microscope employs a 375 nm diode laser with patterned light scanning controlled through two 1-axis galvanometers and an acousto-optic modulator (AOM).

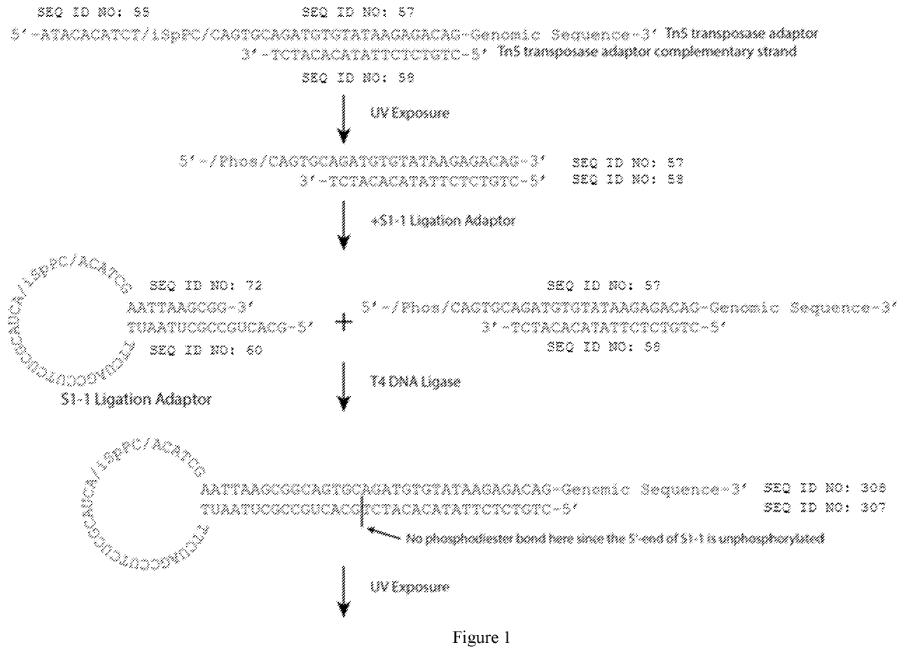

FIG. 1 schematically shows the process of adding an exemplary Ligation Anchor Sequence and an exemplary Transposase Recognition Sequence to the 5′ end of the target nucleic acid molecule (“Genomic Sequence”) of interest and two subsequent ligation-cleavage cycles with exemplary Ligation Adaptors to tag the target nucleic acid molecule with a first barcode and a second barcode. After the genomic DNA is tagged with the desired barcode(s), the cells of the tissue sample or array are then homogenized and the nucleic acid molecules extracted and subjected to nucleic acid sequencing, e.g., high-throughput sequencing.

In the methods described herein, an initial ligation adaptor sequence (“Ligation Anchor Sequence”) and a Transposase Recognition Sequence are attached to the 5′ ends of target nucleic acid molecules of interest, i.e., nucleic acid molecules in the transcriptomes of one or more target cells of interest when the one or more target cells are intact and form part of a tissue sample (e.g., a histological tissue sample) or a spatial array of cells via a Transposase Adaptor.

The Transposase Adaptor comprises a random sequence linked to the Ligation Anchor Sequence via a PC Linker linking the 3′ end of the random sequence to the 5′ end of the Ligation Anchor Sequence, and the 3′ end of the Ligation Anchor Sequence has a Transposase Recognition Sequence whose 3′ end is attached to the 5′ end of the nucleic acid molecules to be barcoded. The random sequence prevents or inhibits unintentional nucleic acid hybridization and ligation to the Ligation Anchor Sequence when the PC Linker is intact. That is, the random sequence upstream of the PC Linker blocks the 5′ end of the Ligation Anchor Sequence from being linked to another sequence when the PC Linker is intact. Thus, the term “PC Blocker” refers to an intact PC Linker having a structure that blocks another nucleic acid molecule from being ligated to a given ligation adaptor sequence (including initial ligation adaptor sequences). The sequence of the random sequence may be any arbitrary nucleic acid sequence. In some embodiments, the random sequence is about 6-15 bases, preferably about 7-14 bases, more preferably about 8-13 bases, even more preferably about 9-11 bases, and most preferably about 10 bases long. The PC Linker may be any moiety that covalently links the 3′ end of the random sequence to the 5′ end of the Ligation Anchor Sequence, which linkage is cleaved upon exposure to a given wavelength of light and leaves a phosphate group on the 5′ end of the Ligation Anchor Sequence upon cleavage. Preferably, the PC Linker is a 1-(2-nitrophenyl)ethyl phosphate ester group, e.g., 1-(2-nitro-5-((4-oxidobutanamido)methyl)phenyl)ethyl phosphate group (“iSpPC” commercially available from Integrated DNA Technologies (IDT)). The Transposase Recognition Sequence is selected based on the particular transposase that will be used, e.g., if the transposase to be used is a Tn5 transposase, then a Tn5 recognition sequence is used.

After adding the Ligation Anchor Sequence and Transposase Recognition Sequence to the 5′ end of target nucleic acid molecules, Ligation Adaptors are then used to add barcodes to the target nucleic acid molecules. Ligation Adaptors comprise the following in the 5′ to 3′ direction: a ligation adaptor reverse complement sequence; a barcode reverse complement sequence; a hairpin loop sequence; a PC Linker having a phosphate group; a ligation adaptor sequence; and a barcode sequence. The sequence of the hairpin loop sequence may be any nucleic acid sequence known in the art to form the loop portion of a hairpin loop structure. See, e.g., Moody (2004) “Stability in Nucleic Acid Hairpins: 1. Molecular Determinants of Cooperativity and 2. Linkage Between Proton Binding and Folding” Thesis, Penn State University. A variety of methods for identifying sequences that form hairpin loop structures are known in the art. See, e.g., Gorodkin, et al., (2001) Nucleic Acids Res. 29(10): 2135-44.

The ligation adaptor reverse complement sequence has a sequence that is the reverse complement of the preceding ligation adaptor sequence that was attached to the 5′ end of the target nucleic acid molecule being barcoded. The barcode reverse complement sequence has a sequence that is the reverse complement of the given barcode sequence. The hairpin loop sequence may be any desired sequence so long as it does not hybridize to itself. The hairpin loop sequence is preferably about 15-25 bases, more preferably about 18-22 bases, and most preferably about 19-21 bases long. In some embodiments, the hairpin loop sequence is about 15-25 bases long. In some embodiments, the hairpin loop sequence is TTCUAGCCUTCUCGCAUCA (SEQ ID NO: 53). The PC Linker may be any moiety that covalently links the 3′ end of the hairpin loop sequence to the 5′ end of the ligation adaptor sequence, which linkage is cleaved upon exposure to a given wavelength of light and leaves a phosphate group on the 5′ end of the ligation adaptor sequence upon cleavage. Preferably, the PC Linker is a 1-(2-nitrophenyl)ethyl phosphate ester group, e.g., iSpPC, which is commercially available from Integrated DNA Technologies (IDT)). The ligation adaptor sequence may be any desired sequence of about 6-10 bases. The barcode sequence may be any desired sequence. The barcode sequence is about 5-15 bases, preferably about 6-14 bases, more preferably about 7-13 bases, even more preferably about 8-12 bases, and most preferably about 9-11 bases long. Because of the barcode sequence and the barcode reverse complement sequence, the Ligation Adaptor forms a hairpin loop structure where the barcode sequence and the barcode reverse complement sequence form the stem and the hairpin loop sequence, PC Linker, and the ligation adaptor sequence form the loop. The hairpin loop structure prevents barcodes from being incorporated (by ligation) onto the 5′ end of target nucleic acid molecules when the PC Linker is intact. When the PC Blocker is exposed to a light wavelength specific to the given PC Linker, the PC Linker is cleaved and thereby results in the 5′ end of the ligation adaptor sequence having a phosphate group that is available for ligation with the 3′ end of a given barcode sequence.

Hundreds of thousands to millions of ligation reactions may be controlled simultaneously by illuminating a tissue sample or array of cells with light in a predefined pattern using, e.g., a computer guided light source or a photomask for a given ligation-cleavage cycle. Subsequent ligation-cleavage cycles may employ the same or different predefined pattern of light to place specific barcodes on one or more of the nucleic acid molecules that were subjected to the previous ligation-cleavage cycle, thereby giving the nucleic acid molecules of a given cell or cells at a specific location in the tissue sample or array of cells a unique barcode or a unique pattern of barcodes that is indicative of location of the cell(s) from which the nucleic acid molecules were derived.

As an example, assume a tissue sample is sectioned into 10 rows and 10 columns (a “10×10 array”). One section of the array may be a single cell or a plurality of cells. The entire array, i.e., tissue sample is treated with a Transposase Adaptor or Ligation Adaptor. Then light is directed to only a given section of the array to thereby expose one section or some sections of the array. Only the nucleic acid molecules derived from cells located in the section(s) exposed to light with have an anchor or ligation adaptor sequence resulting from the PC Linker being cleaved the light exposure. As such, only those nucleic acid molecules will hybridize and ligate with a subsequent Ligation Adaptor having a given barcode. Thus, the given barcode when present on a nucleic acid molecule is a Spatial Barcode as it indicates that the nucleic acid molecule must have originated from a cell that was located in the section of the array that was exposed to light.

As another example, assume a tissue sample is sectioned into 10×10 array and the entire tissue sample is treated with the same Transposase Adaptor and exposed to a first light treatment such that all the nucleic acid molecules of all the cells of the tissue sample will have the same Ligation Anchor Sequence. As such, the nucleic acid molecules of all the cells in the tissue sample will have a first barcode after a first Ligation Adaptor as added thereto via a first cycle of ligation. However, for each subsequent ligation-cleavage cycle with a Ligation Adaptor having the same or different barcode sequence, “n−1” sections of the array (where “n” is the number of sections that were exposed to light in the prior cleavage cycle) are exposed to light and this process is repeated until n=1. As a result, no two sections will yield nucleic acid molecules having the same number or pattern of barcodes. Thus, the number or pattern of barcodes ligated to a given nucleic acid molecule is a Spatial Barcode as such is indicative of the particular section of the array from which it was derived.

As another example, assume a tissue sample is sectioned into 10×10 array and each section of the array is treated to have a unique anchor or ligation adaptor sequence by way of, e.g., unique Transposase Adaptors. Each given unique anchor or ligation adaptor sequence specifically hybridizes and ligates with a unique Ligation Adaptor, i.e., a Ligation Adaptor having a ligation adaptor reverse complement sequence that is specific for the given unique anchor or ligation adaptor sequence and a unique barcode sequence. Each unique anchor or ligation adaptor sequence is hybridized and ligated to its respective Ligation Adaptor thereby resulting in the nucleic acid molecules derived from each section of the array having a Spatial Barcode, i.e., a unique barcode sequence.

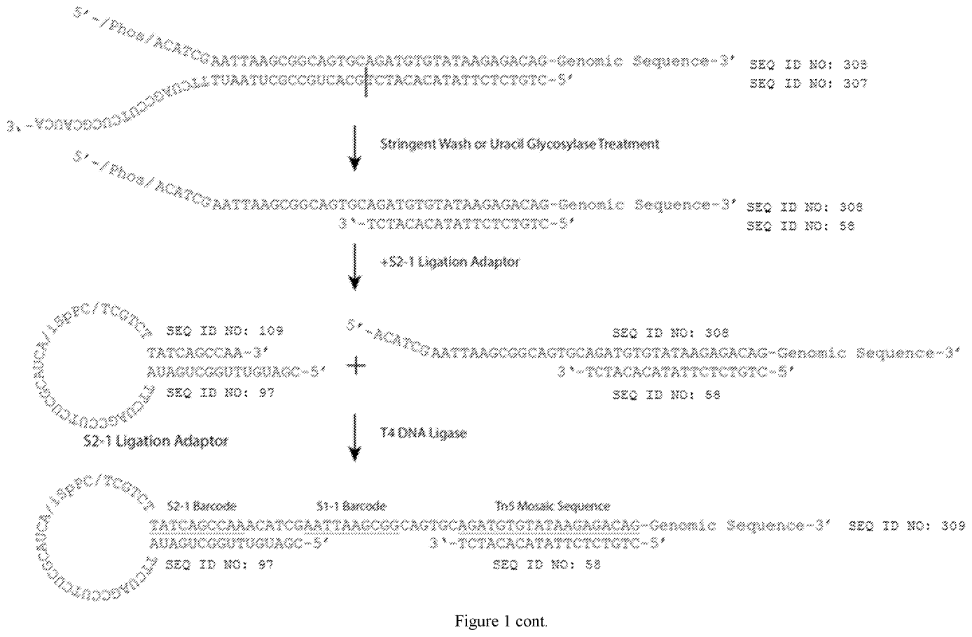

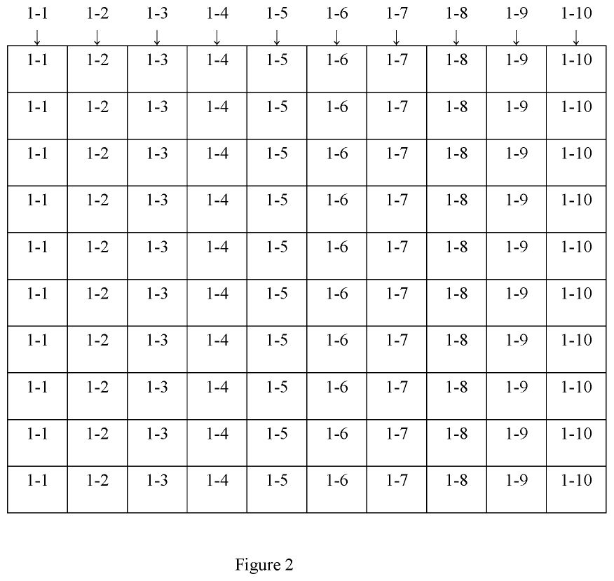

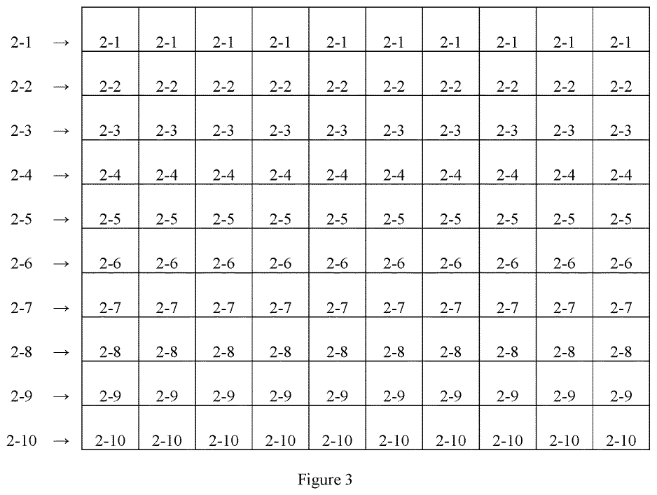

As another example, assume a tissue sample is sectioned into 10×10 array and the entire tissue sample is treated with the same Transposase Adaptor or the same Ligation Adaptor such that the sections will have the same anchor or ligation adaptor sequence upon exposure to light. Then, for a given round of ligation-cleavage, Ligation Adaptors having different barcode sequences are employed. For example, a first set of 10 Ligation Adaptors having the same ligation adaptor reverse complement sequence that is specific for the preceding anchor or ligation adaptor sequence but having different barcode sequences are used in combination with a second set of 10 Ligation Adaptors having the same ligation adaptor reverse complement sequence that is specific for the adaptor sequence of the first set and having barcode sequences that are the same or different from the barcodes of the first set. As schematically shown in FIG. 2, each column in the 10×10 array is treated with a Ligation Adaptor belonging to a given set of Ligation Adaptors, e.g., cells in the first column of the 10×10 array are treated with Ligation Adaptors of the first set (“1-”) having a first barcode sequence (“1”), cells in the second column are treated with Ligation Adaptors of the first set (“1-”) having a second barcode sequence (“2”), etc. The entire array is then exposed to a first light treatment or, alternatively, the cells in the columns are sequentially treated and exposed. Then, as schematically shown in FIG. 3, the rows of the 10×10 array are treated with a different set of Ligation Adaptors, which may have the same or unique barcode sequences. For example, the first row of the array is treated with Ligation Adaptors of the second set (“2-”) having the first barcode sequence (“1”), cells in the second row are treated with Ligation Adaptors of the second set (“2-”) having the second barcode sequence (“2”), etc. The entire array is then exposed to a second light treatment or, alternatively, the cells in the rows are sequentially treated and exposed. As shown in FIG. 4, each section of the array will have been treated with a different combination of Ligation Adaptors thereby resulting in the nucleic acid molecules of those sections having a different combination of barcodes attached thereto. This process may be repeated by columns and rows, randomly, or intentionally to specific sections of the array to add additional barcode sequences to nucleic acid molecule as desired. Thus, the combination of different barcode sequences acts as a Spatial Barcode which indicates the location in the array from which the nucleic acid molecule was derived.

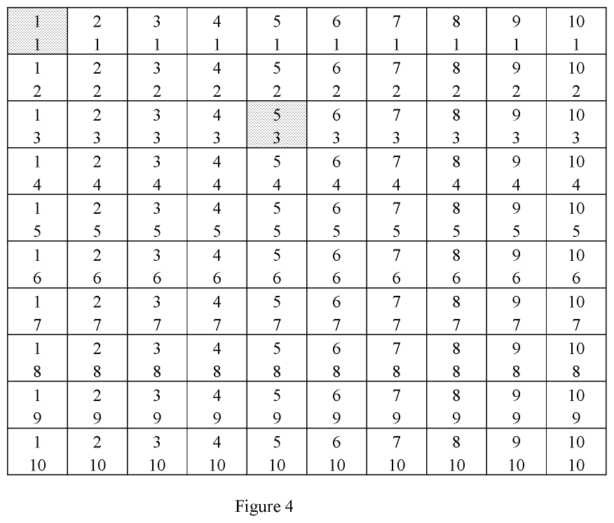

These different pi-seq tagging methods may be used alone or in combination. For example, a computer guided light source or a photomask may be used in combination with one or more sets of Ligation Adaptors having different barcodes. This is schematically shown in FIG. 5. As another example, a plurality of barcodes may be sequentially ligated to a given section of an array by sequential ligation-cleavage cycles performed on the same given section, or by sequential ligation-cleavage cycles whereby a different pattern of light exposure is used for each cycle.

Because the Spatial Barcodes are directly incorporated into sequencing libraries by ligation while the cells are intact and are still part of a tissue sample or an array of cells, the nucleic acid need not be individually extracted from each cell separately. Instead, the total nucleic acid from the tissue sample or array of cells may be extracted together in the same extraction step and the spatial information provided via the Spatial Barcodes will be maintained. That is, the location from which a nucleic acid molecule was derived in tissue sample or array of cells is readily retrievable via the Spatial Barcode appended thereto.

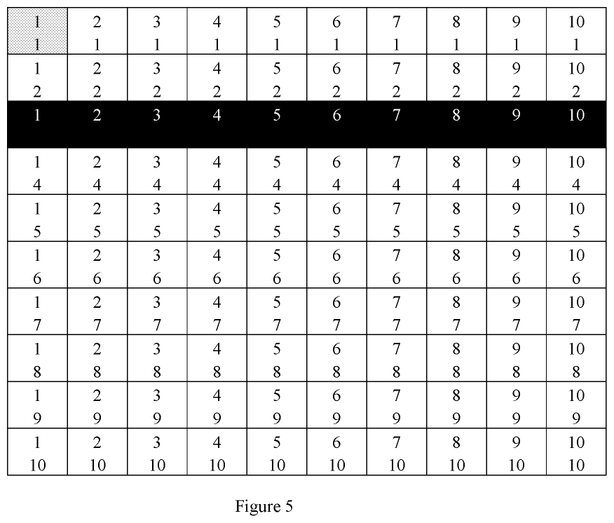

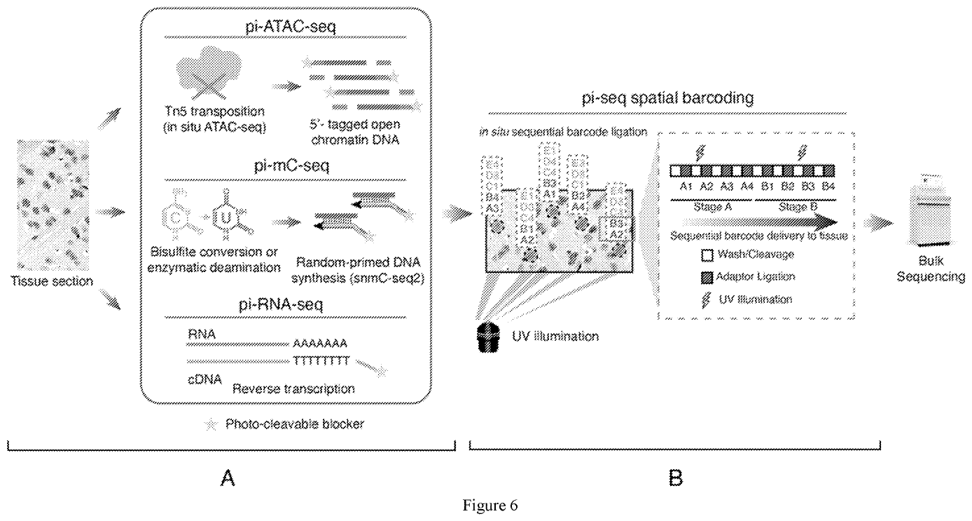

The pi-seq methods described herein may readily be applied to ATAC-seq, mC-seq, and RNA-seq methods in the art. The combined application of pi-seq to these methods are referred to herein as “pi-ATAC-seq”, “pi-mC-seq”, and “pi-RNA-seq” are schematically shown in FIG. 6. As schematically shown in FIG. 7, Pi-ATAC-seq and pi-mC-seq produce genomic DNA tagged with Spatial Barcodes and pi-RNA-seq produces cDNA tagged with Spatial Barcodes.

The pi-seq methods allow the nucleic acid molecules of a single cell to be labeled with a Spatial Barcode specific for the given single cell's location in the tissue sample or array of cells by the use of adaptors (Transposase Adaptors and/or Ligation Adaptors) having a PC Blocker and ligation-cleavage reactions controlled by light, e.g., UV light, which prevents or inhibits “barcode collision”. The use of sets of different barcode sequences (each being about 8-12, preferably about 10 bp in length), e.g., a set of about 10-12 different barcode sequences for each ligation-cleavage cycle prevents “barcode crossover” caused by sequencing errors.

As exemplified herein, for each ligation-cleavage cycle that ligates a barcode to a target nucleic acid molecule of interest, a set of about 10 to about 12 Ligation Adaptors, each having a distinct barcode sequence is used. Prior to introduction of the Ligation Adaptors, an epifluorescence microscope, one-photon laser scanning microscope, or a two-photon scanning microscope is used to expose a cell or cells of interest to light and thereby deprotect the cell(s) and allow the given Ligation Adaptor to be ligated to the nucleic acid molecules of the deprotected cells. After each step of cleaving the given PC Blocker the 3′ to 5′ portion having the barcode reverse complement sequence is removed, e.g., by a stringent washing step or treatment with uracil glycosylase. For example, as shown in part B of FIG. 6, sections of the tissue or array of cells that are to be tagged with Barcode B3 are deprotected by UV illumination prior to the addition of the Ligation Adaptor having the Barcode B3 sequence. Pi-seq may be used to distinctly label the nucleic acid molecules of 10,000 individual cells a tissue section using only 4 ligation-cleavage cycles with 10 unique barcode sequences to result in 10,000 different Spatial Barcodes (i.e., the unique combinations of 4 of 10 of the individual barcodes, e.g., 1+1+1+1, 1+4+6+7, 10+4+2+9, etc.) (10⁴=10,000). to tag the nucleic acid molecules of more than 10,000 cells, each having a unique Spatial Barcode, more than 10 unique barcode sequences may be employed in a given set of Ligation Adaptors and/or additional ligation-cleavage cycles may be employed so that each Spatial Barcode comprises more than 4 individual barcode sequences, e.g., 5, 6, 7, etc. individual barcode sequences.

Regional and Single-Cell pi-ATAC-seq in Cortical Neurons

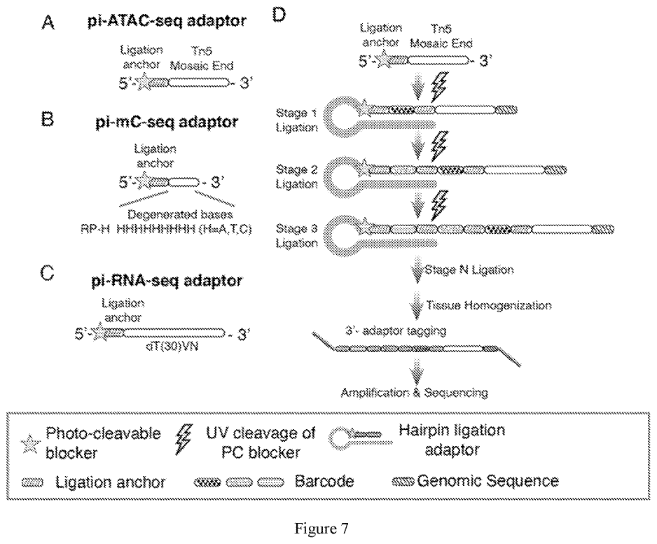

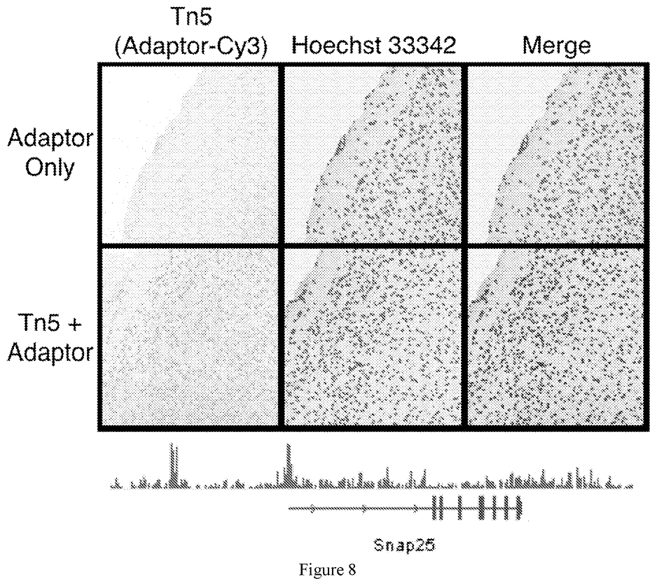

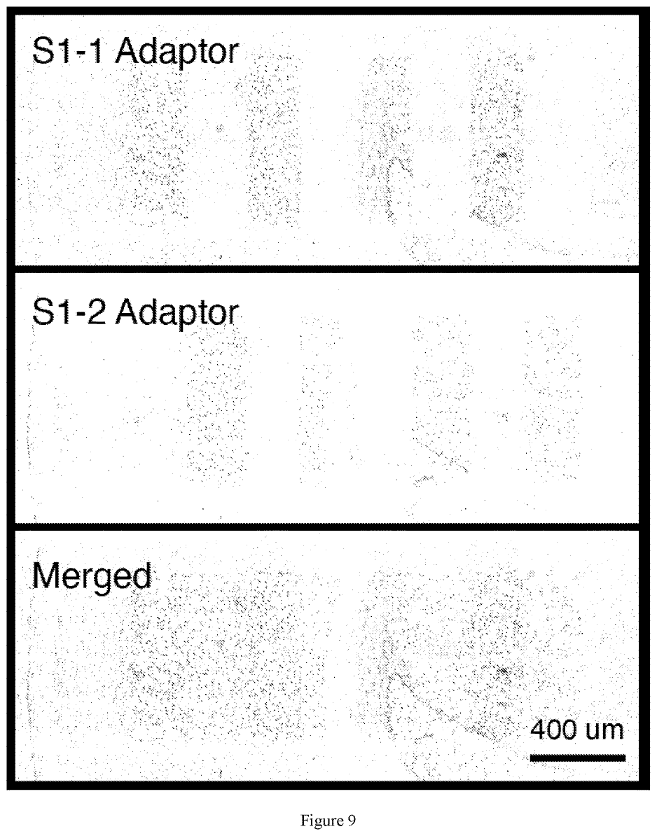

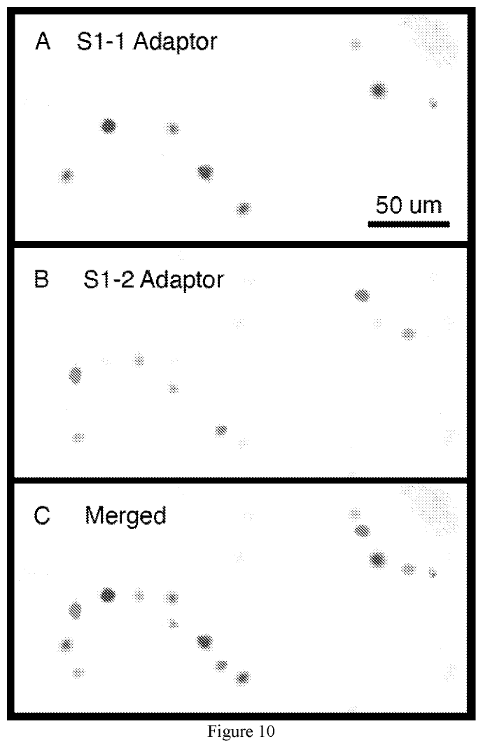

In situ indexing experiments following the tagging of open chromatin regions using Tn5 transposase were performed. 10 μm mouse brain tissue slices were fixed and permeabilized with 0.2% NP40 and then treated with hyperactive Tn5 transposase (see, e.g., Fang, et al. (2021) Nat Commun. 12 (1): 1337) loaded with a Transposase Adaptor containing a Transposase Recognition Sequence for Tn5 transposition and a PC Blocker. As shown in FIG. 8, in situ Tn5 transposition can effectively insert fluorescent adaptors into nuclei. Furthermore, an in situ generated ATAC-seq library was successfully sequenced on Illumina's HISEQ 4000 platform. The library showed chromatin accessibility peaks at promoters and upstream enhancers such as for pan-neuronal marker gene Snap25 (see bottom of FIG. 8). The feasibility of spatial barcoding by sequential ligation of barcode sequences onto the 5′-phosphate end that is released upon cleavage by exposure to light was tested. As shown in FIG. 9, panel A, a mouse brain slice was treated with a Transposase Adaptor for Tn5 and then a first set of sections of the mouse brain slice were exposed to light using an epi-fluorescence UV microscope (using a 40× objective) and the nucleic acid molecules of the cells in those exposed sections were ligated to the S1-1 Ligation Adaptor having a first barcode sequence. Then a different set of sections of the mouse brain slice were similarly exposed to light and the nucleic acid molecules of the cells in those exposed sections were ligated to the S1-2 Ligation Adaptor having a second barcode sequence. The first and second barcode sequences were detected using two different fluorescent probes, each specific for one of the barcode sequences (FIG. 9). The first barcode was detected only in the sections that were exposed to light prior to the addition and ligation of the S1-1 Ligation Adaptor and the second barcode was detected only in the sections that were exposed to light just prior to the addition and ligation of the S1-2 Ligation Adaptor. See FIG. 10. This evidences that adaptors having a PC Blocker and exposure to light via, e.g., 2-photon scanning microscopy, may be used to tag the nucleic acid molecules of a single cell in a tissue sample or an array of cells with a highly specific Spatial Barcode. As shown in FIG. 11, pi-seq was combined with immunofluorescence to select and label (with anti-NeuN antibody) individual single cells.

### Spatial Barcodes Provide Highly Specific Spatial Information

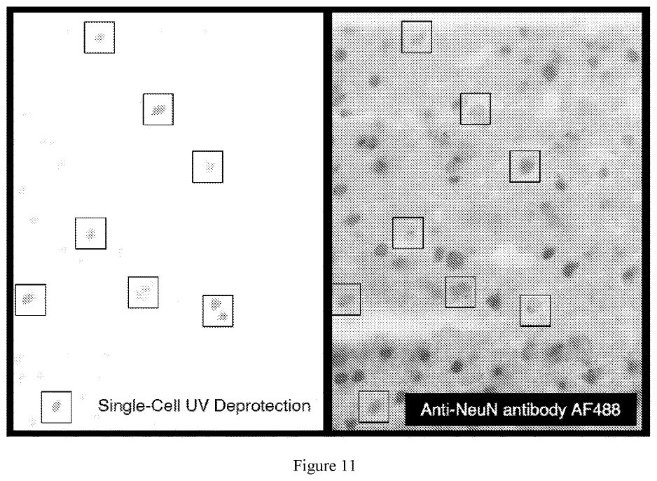

A pi-ATAC-seq library was sequenced following two ligation-cleavage cycles to tag target nucleic acid molecules with two different Spatial Barcodes (Cycle 1 Barcode and Cycle 2 Barcode). Preliminary results indicate that Spatial Barcodes via pi-seq methods provide highly specific and accurate spatial information. Specifically, at least about 88% of sequencing reads provided the correct Spatial Barcode sequence (i.e., the Cycle 2 Barcode sequence+the Cycle 1 Barcode sequence) followed by the Transposase Recognition Sequence (i.e., the given Tn5 mosaic end). See FIG. 12, panel A. Additionally, the generation of pi-ATAC-seq library was strictly dependent on deprotection (i.e., limited to cells that had been exposed to UV light). The sample exposed to UV light (Sample 4) resulted in 181 times more sequencing reads than the sample not exposed to UV. See FIG. 12, panel B. Additionally, Sample 4 compared to Sample 2 of FIG. 12, panel B, indicates that the ligation of the Cycle 2 Barcode sequence is strictly limited to ligation to the ligation adaptor sequence of the Cycle 1 Ligation Adaptor. This suggests a relatively negligible amount of non-specific ligation. Therefore, these results show that the pi-seq methods herein provide highly accurate and fine spatial resolution information at the single cell level which methods are compatible with high-throughput sequencing platforms.

### Spatial Chromatin Accessibility Profiling of Prostate Tumor Tissue

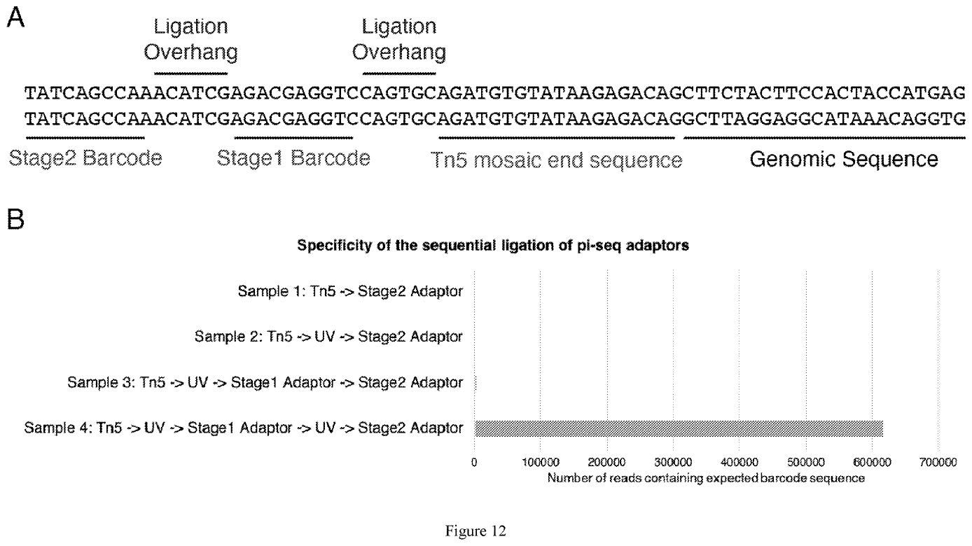

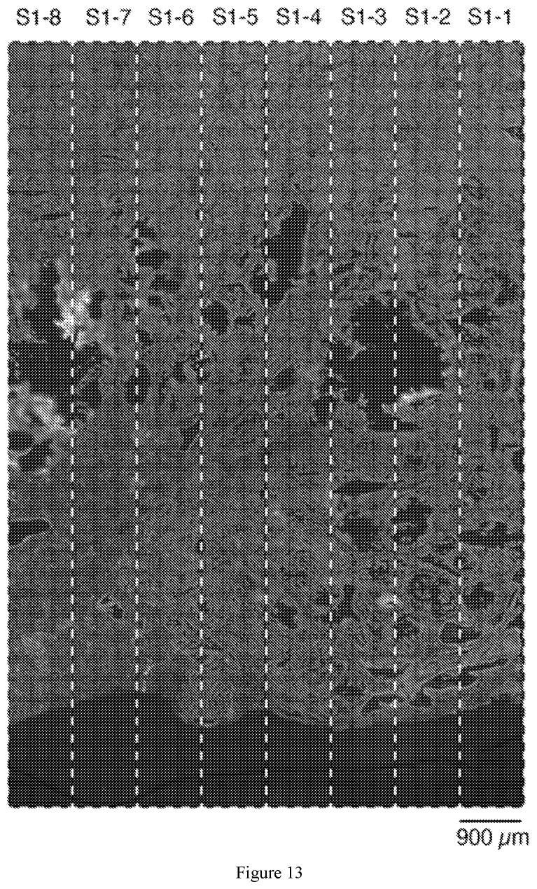

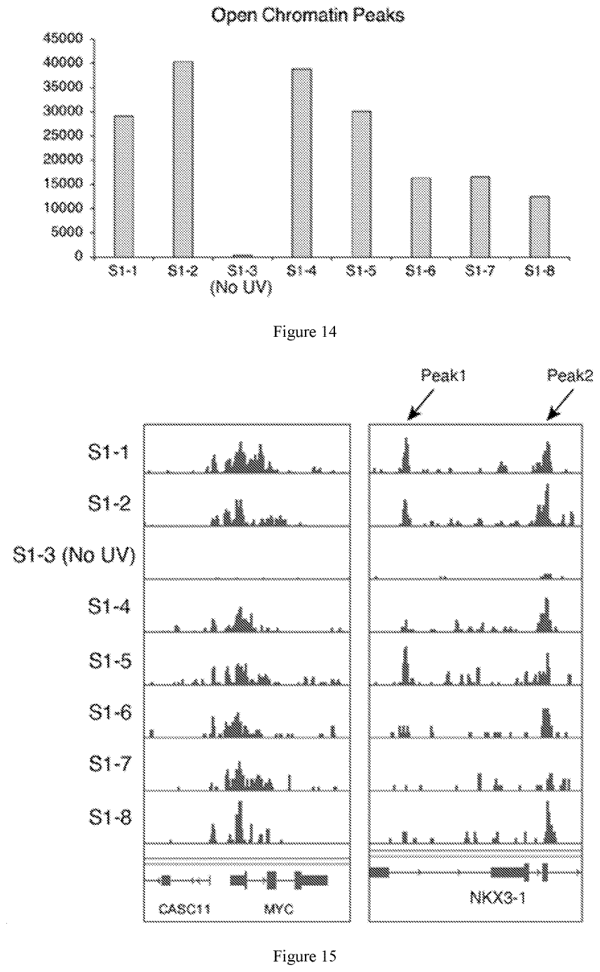

Human prostate tissue was assayed using pi-ATAC-seq using programmed epi-fluorescent UV exposure followed by adaptor ligation reactions to tag the nucleic acid molecules of cells in 8 different sections of the tissue sample as shown in FIG. 13. As shown in FIG. 14, barcode tagging was strictly limited to the given area exposed to UV light. Particularly, there were only 337 open chromatin peaks identified from the section that had not been exposed to UV light, yet treated with a Ligation Adaptor, S1-3, whereas about 10,000 to about 40,410 peaks were identified in the sections that were exposed to UV light. Consistent with the genome-wide result, no open chromatin signal was found at the promoter of oncogene MYC in the no-UV control (S1-3, FIG. 15). This pi-ATAC-seq experiment also identified spatially variable chromatin accessibility such as at an epithelial marker gene NKX3-1 locus, with open chromatin Peak 1 only present in the sections treated with the S1-1, S1-2, and S1-5 Ligation Adaptors, and Peak 2 present in most regions except for a weaker signal in the section treated with the S1-7 Ligation Adaptor (FIG. 15).

Various transposases and their recognition sequences are known in the art. See, e.g., Hickman & Dyda (2015) Microbiol Spectr. 3 (2): MDNA3-0034-2014. In some embodiments, the transposase is an RNase H-like transposase (also known as a DD (E/D) enzyme). In some embodiments, the transposase is selected from Tn5, Mu, RAG, Tn7, TnlO, Vibhar, Tn552, and variants thereof. In some embodiments, is a Tn5 transposase. Exemplary Tn5 transposases include naturally occurring Tn5 transposases, EZTn5™, NexteraV2, TS-Tn5059, and those described in Goryshin & Reznikoff (1998) J. Biol. Chem., 273:7367, U.S. Pat. Nos. 5,925,545, 5,948,622, 5,965,443, 6,140,129, 6,159,736, 6,406,896, 7,083,980, 7,608,434, 9,790,476, 10,035,992, 10,385,323, 10,544,403, US20060294606, US20150291942, US20180171311, and US20200347441. A variety of Tn5 Transposase Recognition Sequences (also referred to as “Tn5 Mosaic Sequences”) are known in the art. See, e.g., Goryshin et al. (1998) PNAS USA 95 (18): 10716-21.

Photocleavable linkers (“PC Linkers”), which are sometimes referred to as “photolabile linkers”, “photocleavable spacers”, “photocleavable modifiers”, etc., are known in the art. See, e.g., U.S. Pat. Nos. 9,000,142, 10,428,379, Olejnik et al. Nucleic Acids Res. 1998 Aug. 1; 26(15):3572-6; Walker, et al. (1989) Photolabile 1-(2-Nitrophenyl)ethyl Phosphate Esters of Adenine Nucleotide Analogues. Synthesis and Mechanism of Photolysis. ChemInform, 20; Ruble, Brittani K., “Design and Application of Photoactivatable Oligonucleotides” (2012) Publicly Accessible Penn Dissertations 572; 2-(2-Nitrophenyl) propyloxycarbonyl (NPPOC) linker (see, e.g., Combinatorial Library Synthesis. Acc Chem Res 1996, 29:123-131, Johnsson, et al. (2011) Bioorg Med Chem Lett 21:3721-3725); o-Nitrobenzylamino Linkers (see, e.g., Rich & Gurwara (1975) Tetrahedron Lett 16:301-304); α-Substituted o-Nitrobenzyl Linkers (see, e.g., Ajayaghosh & Rajasekharan Pillai (1988) Tetrahedron 44:6661-6666); o-Nitroveratryl Linkers (see, e.g., Zehavi & Patchornik (1973) J Am Chem Soc 95:5673-5677); Phenacyl Linkers (see, e.g., Wang, et al. (1976) J Org Chem 41:3258-3261); p-Alkoxyphenacyl Linkers (see, e.g., Bellof, et al. (1985) Chimia 39:317-320), Benzoin Linkers (see, e.g., Rock, et al. (1996) J Org Chem 61:1526-1529); Pivaloyl Linkers (see, e.g., Peukert, et al. (1998) J Org Chem 63:9045-9051) which are herein incorporated by reference. Preferably, the PC Linker is a 1-(2-nitrophenyl)ethyl phosphate ester group, e.g., 1-(2-nitro-5-((4-oxidobutanamido)methyl)phenyl)ethyl phosphate group (“iSpPC” commercially available from Integrated DNA Technologies (IDT)).

As used herein, “barcode collision” refers to two or more nucleic acid molecules that are tagged with the same barcode sequence, yet the nucleic acid molecules were derived from cells that were located at different positions in a tissue sample or array of cells. Barcode collision is common to single-cell profiling techniques because of challenges with treating a single individual cell. In droplet-based methods, barcode collision is caused by the capture of multiple cells in a droplet. In combinatory indexing methods, barcode collision is caused by the capture of the same pair of cells in multiple cycles of indexing reactions.

As used herein, “barcode crossover” refers to the mis-identification of barcodes due to sequencing errors.

As used herein, “ATAC-seq” refers to using Tn5 transposase to covalently insert adaptor into genomic regions associated accessible chromatin and using high-throughput sequencing to identify such open chromatin regions.

As used herein, “mC-seq” refers to using bisulfite conversion and high-throughput sequencing to identify of the location and quantity of 5′-methylcytosine in genomic DNA.

As used herein, “RNA-seq” refers to using high-throughput sequencing to determine the sequence of RNA molecules.

The following examples are intended to illustrate but not to limit the invention.

### EXAMPLES

### Material and Methods

### Custom DNA Oligos

In the sequences below, “/PC Linker/” refers to a PC Linker that links the upstream sequence with the downstream sequence. In the experiments described herein, the iSpPC spacer (obtained from IDT) was used as an exemplary PC Linker.

Initial Tagging with Ligation Anchor Sequence and Transposase Recognition Sequence:

Exemplary Tn5 Transposase Adaptor
(bold font indicates the Ligation
Anchor Sequence,
and italics indicates the Transposase
Recognition Sequence (SEQ ID NO: 56)):
ATACACATCT/PC Linker/CAGTGCAGATGTGTATAAGAGACAG
(the sequence upstream of the PC Linker
can be any random sequence, shown above is
SEQ ID NO: 55, and the sequence after
the PC Linker is SEQ ID NO: 57).
Exemplary Tn5 Transposase Adaptor complementary
strand:
/5Phos/ctgtctcttatacacatct
(the sequence after “5Phos” is SEQ ID NO: 58)
Exemplary pi-seq generic ssDNA blocker
(pi-Blocker):
ctgtctcttatacacatct (SEQ ID NO: 58)

The following are exemplary sets of Ligation Adaptors that can be used in combination. The lowercase bold font indicates the ligation adaptor reverse complement sequence for the ligation adaptor sequence of the prior ligation-cleavage cycle. The lowercase double underlined font indicates the barcode reverse complement sequence. The uppercase bold font indicates a ligation adaptor sequence. The uppercase underlined font indicates the barcode sequence. The regular uppercase font indicates the loop sequence.

### Ligation-Cleavage Cycle S1:

Exemplary S1-prep Ligation Adaptor:
(SEQ ID NO: 59)
GCACTGTCGGTTACTATTCTAGCCTTCTCGCATCATACATCGTAGTAACCGA
Exemplary S1 Ligation Adaptors:
S1-1 Ligation Adaptor:
gcacugccgcutaautTTCUAGCCUTCUCGCAUCA/PC Linker/ACATCGAATTAAGCGG
S1-2 Ligation Adaptor:
gcacuggaccucgucuTTCUAGCCUTCUCGCAUCA/PC Linker/ACATCGAGACGAGGTC
S1-3 Ligation Adaptor:
gcacugagccuautagTTCUAGCCUTCUCGCAUCA/PC Linker/ACATCGCTAATAGGCT
S1-4 Ligation Adaptor:
gcacugaucgguaggcTTCUAGCCUTCUCGCAUCA/PC Linker/ACATCGGCCTACCGAT
S1-5 Ligation Adaptor:
gcacugaccugauaagTTCUAGCCUTCUCGCAUCA/PC Linker/ACATCGCTTATCAGGT
S1-6 Ligation Adaptor:
gcacugccuauaugcaTTCUAGCCUTCUCGCAUCA/PC Linker/ACATCGTGCATATAGG
S1-7 Ligation Adaptor:
gcacuggaucaggugcTTCUAGCCUTCUCGCAUCA/PC Linker/ACATCGGCACCTGATC
S1-8 Ligation Adaptor:
gcacugucacgaguacTTCUAGCCUTCUCGCAUCA/PC Linker/ACATCGGTACTCGTGA
S1-9 Ligation Adaptor:
gcacugugcauaugugTTCUAGCCUTCUCGCAUCA/PC Linker/ACATCGCACATATGCA
S1-10 Ligation Adaptor:
gcacugguauaguacgTTCUAGCCUTCUCGCAUCA/PC Linker/ACATCGCGTACTATAC
S1-11 Ligation Adaptor:
gcacugagacaacacuTTCUAGCCUTCUCGCAUCA/PC Linker/ACATCGAGTGTTGTCT
S1-12 Ligation Adaptor:
gcacuguaggcaaccgTTCUAGCCUTCUCGCAUCA/PC Linker/ACATCGCGGTTGCCTA

In the above S1 Ligation Adaptors:
a) The entire sequence before the PC Linkers, in order from S1-1 to S1-12, are SEQ ID NO: 60 to SEQ ID NO: 71;
b) The entire sequence after the PC Linkers, in order from S1-1 to S1-12, are SEQ ID NO: 72 to SEQ ID NO: 83;
c) The lowercase double underlined sequences, i.e., the barcode reverse complement sequences, in order from S1-1 to S1-12, are SEQ ID NO: 84 to SEQ ID NO: 95;
d) The regular uppercase font, i.e., the loop sequence, is SEQ ID NO: 53;
e) The sequence in uppercase underlined font, i.e., the barcode sequences, in order from S1-1 to S1-12, are: SEQ ID NO: 1 to SEQ ID NO: 12.

### Ligation-Cleavage Cycle S2:

Exemplary S2-prep Ligation Adaptor:
(SEQ ID NO: 96)
CGATGTTAACAGTGGTTTCTAGCCTTCTCGCATCATCGTCTACCACTGTTA
Exemplary S2 Ligation Adaptors:
S2-1 Ligation Adaptor:
cgaugutuggcugauaTTCUAGCCUTCUCGCAUCA/PC Linker/TCGTCTTATCAGCCAA
S2-2 Ligation Adaptor:
cgaugugauggcutggTTCUAGCCUTCUCGCAUCA/PC Linker/TCGTCTCCAAGCCATC
S2-3 Ligation Adaptor:
cgaugutgaggcgaucTTCUAGCCUTCUCGCAUCA/PC Linker/TCGTCTGATCGCCTCA
S2-4 Ligation Adaptor:
cgauguccutcagagcTTCUAGCCUTCUCGCAUCA/PC Linker/TCGTCTGCTCTGAAGG
S2-5 Ligation Adaptor:
cgauguagaggccaagTTCUAGCCUTCUCGCAUCA/PC Linker/TCGTCTCTTGGCCTCT
S2-6 Ligation Adaptor:
cgauguautccgaugaTTCUAGCCUTCUCGCAUCA/PC Linker/TCGTCTTCATCGGAAT
S2-7 Ligation Adaptor:
cgaugutauacggugaTTCUAGCCUTCUCGCAUCA/PC Linker/TCGTCTTCACCGTATA
S2-8 Ligation Adaptor:
cgaugutgagguaacaTTCUAGCCUTCUCGCAUCA/PC Linker/TCGTCTTGTTACCTCA
S2-9 Ligation Adaptor:
cgaugutuaggccutcTTCUAGCCUTCUCGCAUCA/PC Linker/TCGTCTGAAGGCCTAA
S2-10 Ligation Adaptor:
cgaugutcgautcguaTTCUAGCCUTCUCGCAUCA/PC Linker/TCGTCTTACGAATCGA
S2-11 Ligation Adaptor:
cgaugutuaccautcgTTCUAGCCUTCUCGCAUCA/PC Linker/TCGTCTCGAATGGTAA
S2-12 Ligation Adaptor:
cgauguccuautgcggTTCUAGCCUTCUCGCAUCA/PC Linker/TCGTCTCCGCAATAGG

In the above S2 Ligation Adaptors:
a) The entire sequence before the PC Linkers, in order from S2-1 to S2-12, are SEQ ID NO: 97 to SEQ ID NO: 108;
b) The entire sequence after the PC Linkers, in order from S2-1 to S2-12, are SEQ ID NO: 109 to SEQ ID NO: 120;
c) The lowercase double underlined sequences, i.e., the barcode reverse complement sequences, in order from S2-1 to S2-12, are SEQ ID NO: 121 to SEQ ID NO: 132;
d) The regular uppercase font, i.e., the loop sequence, is SEQ ID NO: 53;
e) The sequence in uppercase underlined font, i.e., the barcode sequences, in order from S2-1 to S2-12, are: SEQ ID NO: 13 to SEQ ID NO: 24.

### Ligation-Cleavage Cycle S3:

Exemplary S3-prep Ligation Adaptor:
(SEQ ID NO: 133)
AGACGAATAGCGGTGCTTCTAGCCTTCTCGCATCACTCTGTGCACCGCTAT
Exemplary S3 Ligation Adaptors:
S3-1 Ligation Adaptor:
agacgaaccucuaacaTTCUAGCCUTCUCGCAUCA/PC Linker/CTCTGTTGTTAGAGGT
S3-2 Ligation Adaptor:
agacgaaaugaggaacTTCUAGCCUTCUCGCAUCA/PC Linker/CTCTGTGTTCCTCATT
S3-3 Ligation Adaptor:
agacgacuaauaccagTTCUAGCCUTCUCGCAUCA/PC Linker/CTCTGTCTGGTATTAG
S3-4 Ligation Adaptor:
agacgautggaggccuTTCUAGCCUTCUCGCAUCA/PC Linker/CTCTGTAGGCCTCCAA
S3-5 Ligation Adaptor:
agacgaucauagccgaTTCUAGCCUTCUCGCAUCA/PC Linker/CTCTGTTCGGCTATGA
S3-6 Ligation Adaptor:
agacgauauggcucgcTTCUAGCCUTCUCGCAUCA/PC Linker/CTCTGTGCGAGCCATA
S3-7 Ligation Adaptor:
agacgaccuaugaaggTTCUAGCCUTCUCGCAUCA/PC Linker/CTCTGTCCTTCATAGG
S3-8 Ligation Adaptor:
agacgacgucucaagcTTCUAGCCUTCUCGCAUCA/PC Linker/CTCTGTGCTTGAGACG
S3-9 Ligation Adaptor:
agacgacauacuccucTTCUAGCCUTCUCGCAUCA/PC Linker/CTCTGTGAGGAGTATG
S3-10 Ligation Adaptor:
agacgaaaguguaagcTTCUAGCCUTCUCGCAUCA/PC Linker/CTCTGTGCTTACACTT
S3-11 Ligation Adaptor:
agacgacuacgcacagTTCUAGCCUTCUCGCAUCA/PC Linker/CTCTGTCTGTGCGTAG
S3-12 Ligation Adaptor:
agacgaugcgccacaaTTCUAGCCUTCUCGCAUCA/PC Linker/CTCTGTTTGTGGCGCA

In the above S3 Ligation Adaptors:
a) The entire sequence before the PC Linkers, in order from S3-1 to S3-12, are SEQ ID NO: 134 to SEQ ID NO: 145;
b) The entire sequence after the PC Linkers, in order from S3-1 to S3-12, are SEQ ID NO: 146 to SEQ ID NO: 157;
c) The lowercase double underlined sequences, i.e., the barcode reverse complement sequences, in order from S3-1 to S3-12, are SEQ ID NO: 158 to SEQ ID NO: 169;
d) The regular uppercase font, i.e., the loop sequence, is SEQ ID NO: 53;
e) The sequence in uppercase underlined font, i.e., the barcode sequences, in order from S3-1 to S3-12, are: SEQ ID NO: 25 to SEQ ID NO: 36.

### Ligation-Cleavage Cycle S4:

Exemplary S4-prep Ligation Adaptor
(SEQ ID NO: 170)
ACAGAGTGGTGAGCAATTCTAGCCTTCTCGCATCATACAGGTTGCTCACCA
Exemplary S4 Ligation Adaptors:
S4-1 Ligation Adaptor:
acagagaaucaucgacTTCUAGCCUTCUCGCAUCA/PC Linker/TACAGGGTCGATGATT
S4-2 Ligation Adaptor:
acagagugacucgcguTTCUAGCCUTCUCGCAUCA/PC Linker/TACAGGACGCGAGTCA
S4-3 Ligation Adaptor:
acagagutcutcacagTTCUAGCCUTCUCGCAUCA/PC Linker/TACAGGCTGTGAAGAA
S4-4 Ligation Adaptor:
acagagccgutgcguaTTCUAGCCUTCUCGCAUCA/PC Linker/TACAGGTACGCAACGG
S4-5 Ligation Adaptor:
acagagacgcaguggaTTCUAGCCUTCUCGCAUCA/PC Linker/TACAGGTCCACTGCGT
S4-6 Ligation Adaptor:
acagagccaggcucaaTTCUAGCCUTCUCGCAUCA/PC Linker/TACAGGTTGAGCCTGG
S4-7 Ligation Adaptor:
acagagcgautcacuaTTCUAGCCUTCUCGCAUCA/PC Linker/TACAGGTAGTGAATCG
S4-8 Ligation Adaptor:
acagagcaguacaggcTTCUAGCCUTCUCGCAUCA/PC Linker/TACAGGGCCTGTACTG
S4-9 Ligation Adaptor:
acagagagcutaucacTTCUAGCCUTCUCGCAUCA/PC Linker/TACAGGGTGATAAGCT
S4-10 Ligation Adaptor:
acagagutcgugguagTTCUAGCCUTCUCGCAUCA/PC Linker/TACAGGCTACCACGAA
S4-11 Ligation Adaptor:
acagagaacutgccutTTCUAGCCUTCUCGCAUCA/PC Linker/TACAGGAAGGCAAGTT
S4-12 Ligation Adaptor:
acagagccaucuagaaTTCUAGCCUTCUCGCAUCA/PC Linker/TACAGGTTCTAGATGG

In the above S4 Ligation Adaptors:
a) The entire sequence before the PC Linkers, in order from S4-1 to S4-12, are SEQ ID NO: 171 to SEQ ID NO: 182;
b) The entire sequence after the PC Linkers, in order from S4-1 to S4-12, are SEQ ID NO: 183 to SEQ ID NO: 194;
c) The lowercase double underlined sequences, i.e., the barcode reverse complement sequences, in order from S4-1 to S4-12, are SEQ ID NO: 195 to SEQ ID NO: 206;
d) The regular uppercase font, i.e., the loop sequence, is SEQ ID NO: 53;
e) The sequence in uppercase underlined font, i.e., the barcode sequences, in order from S4-1 to S4-12, are: SEQ ID NO: 37 to SEQ ID NO: 48.

Exemplary Illumina Sequencing Adaptors:

While the Illumina GA adapter sequence, which is italicized (SEQ ID NO: 54), is used to exemplify a universal adaptor for high-throughput sequencing, any universal adaptor for sequencing may be used. The “U” preceding the universal adaptor allows the linearization of a hairpin loop structure via uracil glycosylase and Endonuclease VIII, which cleaves the uracil and breaks the hairpin loop structure. The lowercase bold font indicates a ligation adaptor reverse complement sequence. The uppercase underlined font indicates the barcode sequence. The lowercase double underlined font indicates the barcode reverse complement sequence.

Exemplary S3 Sequencing Adaptors:
S3-1 Sequencing Adaptor:
(SEQ ID NO: 207)
agacgaacctctaacaUTTCCCTACACGACGCTCTTCCGATCTTGTTAGAGGT
S3-2 Sequencing Adaptor:
(SEQ ID NO: 208)
agacgaaatgaggaacUTTCCCTACACGACGCTCTTCCGATCTGTTCCTCATT
S3-3 Sequencing Adaptor:
(SEQ ID NO: 209)
agacgactaataccagUTTCCCTACACGACGCTCTTCCGATCTCTGGTATTAG
S3-4 Sequencing Adaptor:
(SEQ ID NO: 210)
agacgattggaggcctUTTCCCTACACGACGCTCTTCCGATCTAGGCCTCCAA
S3-5 Sequencing Adaptor:
(SEQ ID NO: 211)
agacgatcatagccgaUTTCCCTACACGACGCTCTTCCGATCTTCGGCTATGA
S3-6 Sequencing Adaptor:
(SEQ ID NO: 212)
agacgatatggctcgcUTTCCCTACACGACGCTCTTCCGATCTGCGAGCCATA
S3-7 Sequencing Adaptor:
(SEQ ID NO: 213)
agacgacctatgaaggUTTCCCTACACGACGCTCTTCCGATCTCCTTCATAGG
S3-8 Sequencing Adaptor:
(SEQ ID NO: 214)
agacgacgtctcaagcUTTCCCTACACGACGCTCTTCCGATCTGCTTGAGACG
S3-9 Sequencing Adaptor:
(SEQ ID NO: 215)
agacgacatactcctcUTTCCCTACACGACGCTCTTCCGATCTGAGGAGTATG
S3-10 Sequencing Adaptor:
(SEQ ID NO: 216)
agacgaaagtgtaagcUTTCCCTACACGACGCTCTTCCGATCTGCTTACACTT
S3-11 Sequencing Adaptor:
(SEQ ID NO: 217)
agacgactacgcacagUTTCCCTACACGACGCTCTTCCGATCTCTGTGCGTAG
S3-12 Sequencing Adaptor:
(SEQ ID NO: 218)
agacgatgcgccacaaUTTCCCTACACGACGCTCTTCCGATCTTTGTGGCGCA

In the above S3 Sequencing Adaptors:
a) The sequences in the uppercase underlined font are barcode sequences which, in order from S3-1 to S3-12, are SEQ ID NO: 25 to SEQ ID NO: 36;
b) The lowercase double underlined font are the barcode reverse complement sequences which, in order from S3-1 to S3-12, are SEQ ID NO: 219 to SEQ ID NO: 230; and
c) The italicized sequence is the Illumina GA adapter sequence, which is SEQ ID NO: 54.

Exemplarly S4 Sequencing Adaptors:
S4-1 Sequencing Adaptor:
(SEQ ID NO: 231)
acagagaatcatcgacUTTCCCTACACGACGCTCTTCCGATCTGTCGATGATT
S4-2 Sequencing Adaptor:
(SEQ ID NO: 232)
acagagtgactcgcgtUTTCCCTACACGACGCTCTTCCGATCTACGCGAGTCA
S4-3 Sequencing Adaptor:
(SEQ ID NO: 233)
acagagttcttcacagUTTCCCTACACGACGCTCTTCCGATCTCTGTGAAGAA
S4-4 Sequencing Adaptor:
(SEQ ID NO: 234)
acagagccgttgcgtaUTTCCCTACACGACGCTCTTCCGATCTTACGCAACGG
S4-5 Sequencing Adaptor:
(SEQ ID NO: 235)
acagagacgcagtggaUTTCCCTACACGACGCTCTTCCGATCTTCCACTGCGT
S4-6 Sequencing Adaptor:
(SEQ ID NO: 236)
acagagccaggctcaaUTTCCCTACACGACGCTCTTCCGATCTTTGAGCCTGG
S4-7 Sequencing Adaptor:
(SEQ ID NO: 237)
acagagcgattcactaUTTCCCTACACGACGCTCTTCCGATCTTAGTGAATCG
S4-8 Sequencing Adaptor:
(SEQ ID NO: 238)
acagagcagtacaggcUTTCCCTACACGACGCTCTTCCGATCTGCCTGTACTG
S4-9 Sequencing Adaptor:
(SEQ ID NO: 239)
acagagagcttatcacUTTCCCTACACGACGCTCTTCCGATCTGTGATAAGCT
S4-10 Sequencing Adaptor:
(SEQ ID NO: 240)
acagagttcgtggtagUTTCCCTACACGACGCTCTTCCGATCTCTACCACGAA
S4-11 Sequencing Adaptor:
(SEQ ID NO: 241)
acagagaacttgccttUTTCCCTACACGACGCTCTTCCGATCTAAGGCAAGTT
S4-12 Sequencing Adaptor:
(SEQ ID NO: 242)
acagagccatctagaaUTTCCCTACACGACGCTCTTCCGATCTTTCTAGATGG

In the above S4 Sequencing Adaptors:
a) The sequences in the uppercase underlined font are barcode sequences which, in order from S4-1 to S4-12, are SEQ ID NO: 37 to SEQ ID NO: 48;
b) The lowercase double underlined font are the barcode reverse complement sequences which, in order from S4-1 to S4-12, are SEQ ID NO: 243 to SEQ ID NO: 254; and
c) The italicized sequence is the Illumina GA adapter sequence, which is SEQ ID NO: 54.

### Adaptor Blockers:

The bold font indicates the ligation adaptor sequence that results from the prior ligation-cleavage cycle, the lowercase font indicates the “AGAA” sequence that complements the “TTCU” sequence that is downstream of the ligation adaptor reverse complementary sequence in the exemplified Ligation Adaptors (which increases the Tm of the Adaptor Blockers for more stable hybridization), the double underlined font indicates the last 10 bases of the prep ligation adaptor sequence, and the underlined font indicates the corresponding barcode sequence.

Exemplary S1 Adaptors Blockers:
S1-prep Adaptor Blocker:
(SEQ ID NO: 255)
agaaTAGTAACCGACAGTGC
S1-1 Adaptor Blocker:
(SEQ ID NO: 256)
agaaAATTAAGCGGCAGTGC
S1-2 Adaptor Blocker:
(SEQ ID NO: 257)
agaaAGACGAGGTCCAGTGC
S1-3 Adaptor Blocker:
(SEQ ID NO: 258)
agaaCTAATAGGCTCAGTGC
S1-4 Adaptor Blocker:
(SEQ ID NO: 259)
agaaGCCTACCGATCAGTGC
S1-5 Adaptor Blocker:
(SEQ ID NO: 260)
agaaCTTATCAGGTCAGTGC
S1-6 Adaptor Blocker:
(SEQ ID NO: 261)
agaaTGCATATAGGCAGTGC
S1-7 Adaptor Blocker:
(SEQ ID NO: 262)
agaaGCACCTGATCCAGTGC
S1-8 Adaptor Blocker:
(SEQ ID NO: 263)
agaaGTACTCGTGACAGTGC
S1-9 Adaptor Blocker:
(SEQ ID NO: 264)
agaaCACATATGCACAGTGC
S1-10 Adaptor Blocker:
(SEQ ID NO: 265)
agaaCGTACTATACCAGTGC
S1-11 Adaptor Blocker:
(SEQ ID NO: 266)
agaaAGTGTTGTCTCAGTGC
S1-12 Adaptor Blocker:
(SEQ ID NO: 267)
agaaCGGTTGCCTACAGTGC

In the above S1 Adaptors Blockers:
a) The double underlined sequence is SEQ ID NO: 51; and
b) The sequences in the uppercase underlined font are barcode sequences which, in order from S1-1 to S1-12, are SEQ ID NO: 1 to SEQ ID NO: 12.

Exemplary S2 Adaptors Blockers:
S2-prep Adaptor Blocker:
(SEQ ID NO: 268)
agaaACCACTGTTAACATCG
S2-1 Adaptor Blocker:
(SEQ ID NO: 269)
agaaTATCAGCCAAACATCG
S2-2 Adaptor Blocker:
(SEQ ID NO: 270)
agaaCCAAGCCATCACATCG
S2-3 Adaptor Blocker:
(SEQ ID NO: 271)
agaaGATCGCCTCAACATCG
S2-4 Adaptor Blocker:
(SEQ ID NO: 272)
agaaGCTCTGAAGGACATCG
S2-5 Adaptor Blocker:
(SEQ ID NO: 273)
agaaCTTGGCCTCTACATCG
S2-6 Adaptor Blocker:
(SEQ ID NO: 274)
agaaTCATCGGAATACATCG
S2-7 Adaptor Blocker:
(SEQ ID NO: 275)
agaaTCACCGTATAACATCG
S2-8 Adaptor Blocker:
(SEQ ID NO: 276)
agaaTGTTACCTCAACATCG
S2-9 Adaptor Blocker:
(SEQ ID NO: 277)
agaaGAAGGCCTAAACATCG
S2-10 Adaptor Blocker:
(SEQ ID NO: 278)
agaaTACGAATCGAACATCG
S2-11 Adaptor Blocker:
(SEQ ID NO: 279)
agaaCGAATGGTAAACATCG
S2-12 Adaptor Blocker:
(SEQ ID NO: 280)
agaaCCGCAATAGGACATCG

In the above S2 Adaptors Blockers:
a) The double underlined sequence is SEQ ID NO: 49; and
b) The sequences in the uppercase underlined font are barcode sequences which, in order from S2-1 to S2-12, are SEQ ID NO: 13 to SEQ ID NO: 24.

Exemplary S3 Adaptors Blockers:
S3-prep Adaptor Blocker:
(SEQ ID NO: 281)
agaaGCACCGCTATTCGTCT
S3-1 Adaptor Blocker:
(SEQ ID NO: 282)
agaaTGTTAGAGGTTCGTCT
S3-2 Adaptor Blocker:
(SEQ ID NO: 283)
agaaGTTCCTCATTTCGTCT
S3-3 Adaptor Blocker:
(SEQ ID NO: 284)
agaaCTGGTATTAGTCGTCT
S3-4 Adaptor Blocker:
(SEQ ID NO: 285)
agaaAGGCCTCCAATCGTCT
S3-5 Adaptor Blocker:
(SEQ ID NO: 286)
agaaTCGGCTATGATCGTCT
S3-6 Adaptor Blocker:
(SEQ ID NO: 287)
agaaGCGAGCCATATCGTCT
S3-7 Adaptor Blocker:
(SEQ ID NO: 288)
agaaCCTTCATAGGTCGTCT
S3-8 Adaptor Blocker:
(SEQ ID NO: 289)
agaaGCTTGAGACGTCGTCT
S3-9 Adaptor Blocker:
(SEQ ID NO: 290)
agaaGAGGAGTATGTCGTCT
S3-10 Adaptor Blocker:
(SEQ ID NO: 291)
agaaGCTTACACTTTCGTCT
S3-11 Adaptor Blocker:
(SEQ ID NO: 292)
agaaCTGTGCGTAGTCGTCT
S3-12 Adaptor Blocker:
(SEQ ID NO: 293)
agaaTTGTGGCGCATCGTCT

In the above S3 Adaptors Blockers:
a) The double underlined sequence is SEQ ID NO: 50; and
b) The sequences in the uppercase underlined font are barcode sequences which, in order from S3-1 to S3-12, are SEQ ID NO: 25 to SEQ ID NO: 36.

Exemplary S4 Adaptors Blockers:
S4-prep Adaptor Blocker:
(SEQ ID NO: 294)
agaaTTGCTCACCACTCTGT
S4-1 Adaptor Blocker:
(SEQ ID NO: 295)
agaaGTCGATGATTCTCTGT
S4-2 Adaptor Blocker:
(SEQ ID NO: 296)
agaaACGCGAGTCACTCTGT
S4-3 Adaptor Blocker:
(SEQ ID NO: 297)
agaaCTGTGAAGAACTCTGT
S4-4 Adaptor Blocker:
(SEQ ID NO: 298)
agaaTACGCAACGGCTCTGT
S4-5 Adaptor Blocker:
(SEQ ID NO: 299)
agaaTCCACTGCGTCTCTGT
S4-6 Adaptor Blocker:
(SEQ ID NO: 300)
agaaTTGAGCCTGGCTCTGT
S4-7 Adaptor Blocker:
(SEQ ID NO: 301)
agaaTAGTGAATCGCTCTGT
S4-8 Adaptor Blocker:
(SEQ ID NO: 302)
agaaGCCTGTACTGCTCTGT
S4-9 Adaptor Blocker:
(SEQ ID NO: 303)
agaaGTGATAAGCTCTCTGT
S4-10 Adaptor Blocker:
(SEQ ID NO: 304)
agaaCTACCACGAACTCTGT
S4-11 Adaptor Blocker:
(SEQ ID NO: 305)
agaaAAGGCAAGTTCTCTGT
S4-12 Adaptor Blocker:
(SEQ ID NO: 306)
agaaTTCTAGATGGCTCTGT

In the above S4 Adaptors Blockers:
a) The double underlined sequence is SEQ ID NO: 52; and
b) The sequences in the uppercase underlined font are barcode sequences which, in order from S4-1 to S4-12, are SEQ ID NO: 37 to SEQ ID NO: 48.

### Reagents

2× Transposition Buffer (TB): 66 mM Tris-Ac (pH=7.8), 132 mM KAc, 22 mM Mg(Ac)₂, 32% Dimethylformamide (DMF)
1× T4 DNA Ligase Buffer without ATP: 50 mM Tris-HCl (pH=8), 10 mM MgCl₂, 10 mM DTT
1× T4 DNA Ligase Buffer with PEG6000: 66 mM Tris-HCl (pH=7.6), 10 mM MgCl₂, 1 mM DTT, 1 mM ATP 7.5% Polyethylene glycol (PEG6000)
High-Salt Wash Buffer: 1×DPBS, 30% DMF, 1 mM pyrophosphate
Low-Salt Wash Buffer: 0.1×DPBS, 30% DMF
Tn5 transposition reaction mixture (1×TB buffer, 12.5 ng/μl Tn5 transposase, 125 nM Tn5-PC adaptor, 0.1% Tween-20) incubated at room temperature for 15 min.

### Reagent Preparation

Preparation of double-stranded Tn5 Transposase Adaptor: Tn5 Transposase Adaptor (100 μM) and Tn5 Transposase Adaptor complementary strand (100 μM) were mixed in equal amounts. The mixed DNA oligos were heated to 95° C. for 5 min and slowed (0.1° C. per second) cooled to room temperature using a thermocycler.

### In Situ Tagmentation of Open Chromatin Regions

Fresh mouse brain or prostate tumor tissues were embedded in Optimal Cutting Temperature (OCT) compound and frozen in a slurry of 2-Methylbutane and dry ice. The tissue blocked were slices using a CryoStat to 10 μm sections and mounted on SuperFrost Plus microscope slides and stored at −80° C.

Slides were removed from −80° C. freezer and fixed with 1% formaldehyde in 1×DPBS at room temperature for 10 minutes. After rinsing the tissue section with 1 ml of 1×DPBS for three times, the fixation reaction was quenched with 2 M Glycine at room temperature for 5 min.

The tissue section was rinsed with 1×DPBS for 3 times and then incubated with the permeabilization solution (1×TB buffer, 0.2% IGAPEL-630, 5 μM pi-Blocker) at room temperature for 10 minutes.

The permeabilization solution was replaced with the Tn5 transposition reaction mixture and incubated at 37° C. for 1 hour and then rinsed off with 1×DPBS for three times. The tissue section was stained with SYTO™ Deep Red Nucleic Acid Stain at room temperature for 30 minutes to visualize the nuclei.

### Regional Spatial Barcoding

To enable automated liquid exchange, a liquid flow with a height of 75 μm was mounted on top on the tissue. The top of the flowcell was formed by a #1.5 coverslip (0.17 mm thickness).

At the beginning of each ligation-cleavage cycle, a ligation reaction containing a preparation adaptor (e.g., S1-prep) is applied to the tissue section to block any free 5′-phosphate that resulted from spontaneous cleavage of the PC Linker (by, e.g., ambient light). For the ligation of preparation adaptors, tissue section was conditioned with 1 mL of 1× T4 DNA Ligase Buffer without ATP with a flow rate of 500 μl per min. The ligation of preparation adaptors was performed in 1× T4 DNA Ligase Buffer with PEG6000 containing 50,000 U/μl T4 DNA ligase and 500 nM preparation adaptor. The ligation reaction was incubated at room temperature for 15 minutes. The tissue section was then washed with 1 mL Low-Salt Washing Buffer at a flow rate of 500 μl per min. The tissue section was subsequently washed with 1 mL High-Salt Washing buffer containing 1 μM blocking oligo for the corresponding preparation adaptor.

The following reaction cycle was repeated for each Ligation Adaptor: Epi-fluorescent UV illumination scan using a 40× NA=1.25 objective and a Lumencor SOLA lightsource which generates approximately 30 mW illumination at 380 nm. Tissue section was conditioned with 1.0 mL of 1× T4 DNA Ligase Buffer without ATP with a flow rate of 500 μl per min. 400 μM of ligation reaction containing 20,000,000 units of T4 DNA ligase and 500 nM Ligation Adaptor was introduced into the flowcell and incubated with the tissue section at room temperature for 15 minutes. The tissue section was then washed with 1 mL Low-Salt Washing Buffer at a flow rate of 500 μl per min. The tissue section was subsequently washed with 1 mL High-Salt Washing buffer containing 1 μM Adaptor Blocker for the corresponding Ligation Adaptor.

### Single-Cell Spatial Barcoding

Single cell spatial barcoding was performed using 1-photon or 2-photon scanning microscopy using a 40× NA=1.25 objective. The scanning was performed with approximately 30 mW on-stage laser power targeted to 10 μm×10 μm regions surrounding single nucleus labeled with SYTO™ Deep Red Nucleic Acid Stain. The conditions for adaptor ligation and washing were identical as regional spatial barcoding reactions.

### Tissue Homogenization and Nucleic Acid Sequencing

Tissues were homogenized by gently scraping the tissue off the microscopic slide. The nucleic acid molecules were extracted by Qiagen DNeasy® Blood & Tissue Kits per manufacturer instructions.

The extracted nucleic acid molecules were then 3′-tagged with a sequencing adaptor using Swift Bioscience Adaptase™ or Splint Ligation, followed by PCR amplification of the sequencing library. Sequencing was performed using Illumina's HiSeq 4000, Nextseq 2000 and compatible equipment per manufacturer instructions.

### REFERENCES

The references recited herein are incorporated by reference in their entirety with the exception that, should the scope and meaning of a term conflict with a definition explicitly set forth herein, the definition explicitly set forth herein controls:

All scientific and technical terms used in this application have meanings commonly used in the art unless otherwise specified.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. (canceled)

2. A Ligation Adaptor for tagging a nucleic acid molecule with a barcode sequence, said Ligation Adaptor comprising a ligation adaptor sequence of about 6-10 bases long with a photocleavable linker (PC Linker) having a phosphate group that is linked to one end of the ligation adaptor sequence, and the barcode sequence linked to the other end of the ligation adaptor sequence.

3. The Ligation Adaptor according to claim 2, wherein
the barcode sequence is 8-12 bases, 9-11 bases, or 10 bases long, and optionally the barcode sequence is non-naturally occurring in the genome from which the nucleic acid molecule was obtained;
the barcode sequence is selected from the group consisting of SEQ ID NO: 1 to SEQ ID NO: 52, and complementary and reverse complementary sequences thereof; and optionally the barcode sequence is non-naturally occurring in the genome from which the nucleic acid molecule was obtained; or
the barcode sequence is selected from the group consisting of SEQ ID NO: 1 to SEQ ID NO: 48, and complementary and reverse complementary sequences thereof, and optionally the barcode sequence is non-naturally occurring in the genome from which the nucleic acid molecule was obtained.

4. The Ligation Adaptor according to claim 2, further comprising a hairpin loop sequence of about 15-25 bases long that has one end linked to the PC Linker.

5. The Ligation Adaptor according to claim 4, further comprising a sequence that is a reverse complement of the barcode sequence, said sequence attached to the end of the hairpin loop sequence opposite to the end attached to the PC Linker.

6. The Ligation Adaptor according to claim 5, wherein the hairpin loop sequence is SEQ ID NO: 53.

7. The Ligation Adaptor according to claim 2, wherein the nucleic acid molecule comprises a photocleavable blocker (“PC Blocker”) linked to an initial ligation adaptor sequence, and optionally comprising a sequence that is a reverse complement of the initial ligation adaptor sequence.

8. The Ligation Adaptor according to claim 7, wherein the ligation adaptor sequence and the initial ligation adaptor sequence are each independently selected from the group consisting of CAGTGC, GCACUG, CGAUGU, AGACGA, ACAGAG, and reverse complements thereof.

9. A method of tagging a nucleic acid molecule with a barcode sequence using the Ligation Adaptor according to claim 7, wherein the barcode sequence provides information about the location of a cell in a tissue sample or in an array of cells to which the nucleic acid molecule was derived, said method comprises performing the following steps
(a) cleaving the PC Blocker by selectively exposing the cell to light;
(b) adding the Ligation Adaptor (“first Ligation Adaptor”) to the nucleic acid molecule (“first nucleic acid molecule”) within the cell (“first cell”); and
(c) adding ligase to the cell; and
(d) optionally, repeating steps (a)-(c) one or more times with a subsequent Ligation Adaptor (“first subsequent Ligation Adaptor”), wherein each subsequent Ligation Adaptor comprises a sequence that is the reverse complement of the ligation adaptor sequence of the preceding Ligation Adaptor, and the barcode sequence of each subsequent Ligation Adaptor may be the same or different from the barcode sequence of the preceding Ligation Adaptor; while the cell is intact and remains a part of the tissue sample or the array.

10. The method according to claim 9, wherein selectively exposing the cell to light comprises using a photomask to block other cells in the tissue sample or the array from exposure to light and/or using a laser or a microscope such as an epifluorescence microscope, a one-photon laser scanning microscope, or a two-photon scanning microscope to focus light on the cell.

11. The method according to claim 9, further comprising tagging a second nucleic acid molecule of a second cell, which comprises
(e) performing steps (a)-(c) with a second Ligation Adaptor having a second barcode sequence that is different from the barcode sequence of the first Ligation Adaptor, and
(f) optionally repeating steps (a)-(c) one or more times with a second subsequent Ligation Adaptor, wherein each second subsequent Ligation Adaptor comprises a sequence that is the reverse complement of the ligation adaptor sequence of the preceding second Ligation Adaptor, and the barcode sequence of each second subsequent Ligation Adaptor may be the same or different from the barcode sequence of the preceding second Ligation Adaptor;
while the second cell is intact and remains a part of the tissue sample or the array.

12. The method according to claim 9,
wherein (i) the barcode sequences of the first Ligation Adaptor and the first subsequent Ligation Adaptor(s) are different, (ii) the barcode sequences of the second Ligation Adaptor and the second subsequent Ligation Adaptor(s) are different, or both (i) and (ii);
wherein the PC Linkers of the Ligation Adaptor and the PC Blocker are the same or different; and/or
wherein the nucleic acid molecules of a cell or cells in different sections of the tissue sample or the array are tagged with unique barcode sequences and/or unique combinations of barcode sequences.

13. (canceled)

14. The method according to claim 9, further comprising
providing a Transposase Recognition Sequence downstream of the initial ligation adaptor sequence;
obtaining an extract of all the nucleotide molecules of the cells of the tissue sample or the array after tagging the first and/or second nucleic acid molecules with one or more barcode sequences, and sequencing the nucleic acid molecules having the one or more barcode sequences; and/or
identifying the barcode sequence(s), number of barcode sequences, and/or combination of different barcode sequences ligated to a given nucleic acid molecule and correlating such to the position of the cell in the tissue sample or array that was treated with the particular Ligation Adaptor(s) that would necessarily result in the identified barcode sequence(s), number of barcode sequences, and/or combination of different barcode sequences ligated to the given nucleic acid molecule.

15-17. (canceled)

18. A nucleic acid molecule comprising a barcode sequence selected from the group consisting of SEQ ID NO: 1 to SEQ ID NO: 52, and complementary and reverse complementary sequences thereof; linked to a universal sequencing adaptor and/or a ligation adaptor sequence.

19. The nucleic acid molecule according to claim 18, and further comprising a sequence that is the reverse complement of the barcode sequence.

20. The nucleic acid molecule according to claim 18, wherein a uracil base precedes the universal sequencing adaptor, both of which are flanked by the barcode sequence and the reverse complement of the barcode sequence.

21. The nucleic acid molecule according to claim 18, wherein
the ligation adaptor sequence is selected from the group consisting of CAGTGC, GCACUG, CGAUGU, AGACGA, ACAGAG, and reverse complements thereof; and/or
the universal sequencing adaptor sequence is SEQ ID NO: 54.

22. (canceled)

23. A kit comprising a plurality of Ligation Adaptors according to claim 1.

24. The kit according to claim 23, further comprising a nucleic acid molecule comprising a barcode sequence selected from the group consisting of SEQ ID NO: 1 to SEQ ID NO: 52, and complementary and reverse complementary sequences thereof; linked to a universal sequencing adaptor and/or a ligation adaptor sequence.

25. A composition comprising (a) a mixture of one or more Ligation Adaptors according to claim 1, or (b) a mixture of one or more nucleic acid molecules each independently comprising a barcode sequence selected from the group consisting of SEQ ID NO: 1 to SEQ ID NO: 52 and complementary and reverse complementary sequences thereof, wherein said barcode sequence is linked to a universal sequencing adaptor and/or a ligation adaptor sequence.

26. The Ligation Adaptor according to claim 2, wherein said Ligation Adaptor comprises, from the 5′ to 3′ end, a sequence that is the reverse complement of a ligation adaptor sequence which is linked to a sequence that is the reverse complement of the barcode sequence which is linked to a hairpin loop sequence of about 15-25 bases long which is linked to a photocleavable linker (PC Linker) which has a phosphate group and is linked to the ligation adaptor sequence which is linked to the barcode sequence, and wherein the barcode sequence is selected from the group consisting of SEQ ID NO: 1 to SEQ ID NO: 52, preferably the barcode sequence is selected from the group consisting of SEQ ID NO: 1 to SEQ ID NO: 48.
